(19) United States
(12) Patent Application Publication
(10) Pub. No.: US 2011/0151432 A1
(43) Pub. Date: Jun. 23, 2011

(54) METHODS AND SYSTEMS TO COLLECT AND PREPARE SAMPLES, TO IMPLEMENT, INITIATE AND PERFORM ASSAYS, AND TO CONTROL AND MANAGE FLUID FLOW

(75) Inventors: Thomas M. Zappia, Somerville, MA (US); Brandon T. Johnson, Cambridge, MA (US); Kate Christian, Somerville, MA (US); David Vogler, Rochester, NY (US); David Butz, Groton, MA (US)

(73) Assignee: BOSTON MICROFLUIDICS, Cambridge, MA (US)

(21) Appl. No.: 12/908,829

(22) Filed: Oct. 20, 2010

Related U.S. Application Data
(63) Continuation-in-part of application No. 12/228,081, filed on Jul. 16, 2008.
(60) Provisional application No. 61/253,356, filed on Oct. 20, 2009.
(60) Provisional application No. 61/253,365, filed on Oct. 20, 2009.
(60) Provisional application No. 61/253,373, filed on Oct. 20, 2009.
(60) Provisional application No. 61/253,377, filed on Oct. 20, 2009.
(60) Provisional application No. 61/253,383, filed on Oct. 20, 2009.
(60) Provisional application No. 61/266,019, filed on Dec. 2, 2009.

(51) Int. Cl. C12Q 1/70; G01N 1/00; B01D 35/00; G01N 33/53; C12M 1/00; G01N 33/566; A61B 5/157

(52) U.S. Cl. 435/5; 422/63; 422/500; 422/535; 435/7.1; 435/283.1; 436/501; 436/177; 600/309

(57) ABSTRACT

Methods and systems to related to sample collection, assays, and fluid control and management, including methods and systems to implement hand-held portable assays, to activate an assay system, to collect and prepare samples, to capture antibodies, and to trap or capture gas bubbles. Methods and systems disclosed herein, and portions thereof, may be implemented alone and/or in various combinations with one another.

### CROSS REFERENCE

This application is a continuation-in-part of U.S. Utility patent application Ser. No. 12/228,081, filed Jul. 16, 2008, and claims the benefit of:

U.S. Provisional Application No. 61/253,356, filed Oct. 20, 2009;

U.S. Provisional Application No. 61/253,365, filed Oct. 20, 2009;

U.S. Provisional Application No. 61/253,373, filed Oct. 20, 2009;

U.S. Provisional Application No. 61/253,377, filed Oct. 20, 2009;

U.S. Provisional Application No. 61/253,383, filed Oct. 20, 2009; and

U.S. Provisional Application No. 61/266,019, filed Dec. 2, 2009;

all of which are incorporated herein by reference in their entireties.

### TECHNICAL FIELD

Disclosed herein are methods and systems related to sample collection, assays, and fluid control and management.

### BACKGROUND

Given the great strain on the healthcare work force, the increased prevalence of many common diseases and the substantial delay in treatment caused by remote testing, it has become imperative to develop rapid, easy-to-use automated diagnostic devices and platforms to enable efficient and accurate point-of-care disease detection.

Historic obstacles to point-of-care devices include manufacturing challenges, ease-of-use limitations, and government regulations. Some of these obstacles have been reduced through advances in technology and recognition by governments and other regulatory bodies of the importance of point-of-care testing. However, important considerations, including ease-of-use and accuracy, still render point-of-care tests unsuitable for many healthcare facilities.

Conventional point-of-care diagnostic systems utilize capillary action or test strips, which provide limited ability to perform many diagnostic assays, such as fluidic assays.

Fluidic assays, such as enzyme-linked immuno-sorbent assays (ELISAs), are capable of detecting the presence of many diseases ranging from cancer to diseases like herpes simplex type 2, and generally require relatively few operational steps. However, these steps are typically preformed by trained lab technicians.

### SUMMARY

Disclosed herein are methods and systems to implement hand-held portable assays.

Methods and systems disclosed herein, and portions thereof, may be implemented alone and/or in various combinations with one another.

### BRIEF DESCRIPTION OF THE DRAWINGS/FIGURES

In the drawings, like reference numbers indicate identical or functionally similar elements. Additionally, the leftmost digit(s) of a reference number identifies the drawing in which the reference number first appears.

FIG. 1 is a process flowchart of a method of performing an assay with a substantially self-contained, point-of-care, user-initiated fluidic assay system.

FIG. 2 is a block diagram of a portable, point-of-care, user-initiated fluidic assay system.

FIG. 3 is a perspective view of a portable, point-of-care, user-initiated fluidic assay system 300.

FIG. 4 is a process flowchart of a method of preparing a portable, point-of-care, user-initiated fluidic assay system.

FIG. 5 is a process flowchart of a method of using an assay system prepared in accordance with FIG. 4.

FIG. 6 is a perspective view of another assay system 600, including a cover illustrated in a first position.

FIG. 7 is a cross-sectional view of an assay system 600, including plungers 702, 704, and 706, wherein the cover is illustrated in the second position.

FIG. 8 is another cross-sectional view of assay system 600, wherein plungers 702, 704, and 706 are in corresponding initial or first positions.

FIG. 9 is another cross-sectional view of assay system 600, wherein plungers 702, 704, and 706 are in respective first intermediate positions.

FIG. 10 is another cross-sectional view of assay system 600, wherein plunger 704 is in a second position, and plungers 702 and 704 are in respective second intermediate positions.

FIG. 11 is another cross-sectional view of assay system 600, wherein plungers 702, 704 and 706 are in respective second positions.

FIG. 12 is an expanded cross-sectional view of a portion of assay system 600, including a portion of plunger 706 in the first position corresponding to FIG. 8.

FIG. 13 is another expanded cross-sectional view of a portion assay system 600, including a portion of plunger 706 in the intermediate position corresponding to FIG. 9.

FIG. 14 is another expanded cross-sectional view of a portion of assay system 600, including a portion of plunger 706 in the second position corresponding to FIGS. 10 and 11.

FIG. 15 is a cross-sectional perspective view of another assay system 1500.

FIG. 16 is a cross-sectional perspective view of another assay system 1600.

FIG. 17 is cross-sectional view of a mechanical actuator system.

FIG. 18 is a profile view of another fluidic assay system.

FIG. 19 is a cross-sectional view of the fluidic assay system of FIG. 18.

FIG. 20 is a graphic depiction of an example pathway of an activation post of the fluidic assay system of FIG. 18.

FIG. 21 is a cross-sectional view of a housing portion of the fluidic assay system of FIG. 18, including the activation post pathway of FIG. 20 formed within an inner surface the housing portion.

FIGS. 22A through 22F graphically depict an example sequence of movements within the fluidic assay system of FIG. 18 in response to the activation post pathway of FIG. 21.

FIGS. 23A and 23B are cross-sectional views of a fluid chamber of the fluidic assay system of FIG. 18.

FIG. 24 is a perspective view of a sample collection system.

FIG. 25 is another perspective view of the sample collection system.

FIG. 26A is a cross-sectional view of the sample collection system, wherein a plunger is illustrated in a first position.

FIG. 26B is another cross-sectional view of the sample collection system, wherein the plunger is illustrated in a second position.

FIG. 27A is another perspective view of another sample collection system.

FIG. 27B is a cross-sectional view of the sample collection system of FIG. 27A.

FIG. 28 is a profile view of a bubble trap system.

FIG. 29 is a cross-sectional view of the bubble trap system.

FIG. 30 is an upwardly directed view of an upper portion of the bubble trap system.

FIG. 31A through 31C graphically depict movement of fluid and gas bubbles through fluid channels and collection of gas bubbles.

FIGS. 32A through 32E are additional cross-sectional views of the bubble trap system, to illustrate fluid flow and bubble trapping.

FIG. 33 is an upwardly directed view of an upper portion of another bubble trap system, including multiple interconnected membrane active areas, each including a corresponding bubble termination trap.

FIG. 34 is a cross-sectional side view of a competitive molecule capture system.

FIG. 35 is a cross-sectional side view of another competitive molecule capture system.

FIG. 36 is a cross-sectional perspective view of the competitive molecule capture system of FIG. 35.

In the drawings, the leftmost digit(s) of a reference number may identify the drawing in which the reference number first appears.

### DETAILED DESCRIPTION

Example methods and systems are disclosed herein with respect to point-of-care, user-initiated fluidic assay methods and systems, for illustrative purposes. Methods and systems disclosed herein are not, however, limited to the assay methods and systems disclosed herein. Based on the teachings herein, one skilled in the art will understand that methods and system disclosed herein may be implemented with respect to other assay systems, including diagnostic assays and chemical assays.

An immunoassay is a biochemical test to detect a substance, or measure a concentration of a substance, in a biological sample such as blood, saliva, or urine, using a reaction between an antibody and an antigen specific to the antibody.

An immunoassay may be used to detect the presence of an antigen or an antibody. For example, when detecting an infection, the presence of an antibody against the pathogen may be measured. When detecting hormones such as insulin, the insulin may be used as the antigen.

Accordingly, where a method or system is described herein to detect a primary binding pair molecule using a corresponding second binding pair molecule, it should be understood that the primary binding pair molecule may be an antibody or an antigen, and the second binding pair molecule may be a corresponding antigen or antibody, respectively. Similarly, where a method or system is described herein to detect an antibody or antigen, the method or system may be implemented to detect a corresponding antigen or antibody, respectively.

Immunoassays may also be used to detect potential food allergens and chemicals, or drugs.

Immunoassays include labeled immunoassays to provide a visual indication of a binding pair of molecules. Labeling may include an enzyme, radioisotopes, magnetic labels, fluorescence, agglutination, nephelometry, turbidimetry and western blot.

Labeled immunoassays include competitive and non-competitive immunoassays. In a competitive immunoassay, an antigen in a sample competes with labeled antigen to bind with antibodies. The amount of labeled antigen bound to the antibody site is inversely proportional to the concentration of antigen in the sample. In noncompetitive immunoassays, also referred to as sandwich assays, antigen in a sample is bound to an antibody site. The labeled antibody is then bound to the antigen. The amount of labeled antibody on the site is directly proportional to the concentration of the antigen in the sample.

Labeled immunoassays include enzyme-linked immuno-sorbent assays (ELISA).

In an immunoassay, a biological sample is tested for a presence of a primary binding pair molecule. A corresponding binding pair molecule that is specific to the primary binding pair molecule is immobilized on an assay substrate. The biological sample is contacted to the assay substrate. Any primary binding pair molecules in the biological sample attach to, or are captured by the corresponding binding pair molecules. The primary binding pair molecules are also contacted with labeled secondary binding pair molecules that attach to the primary binding pair molecules. This may be performed subsequent to, prior to, or simultaneously with the contacting of the primary binding pair molecule with the corresponding immobilized binding pair molecule. Un-reacted components of the biological sample and fluids may be removed, or washed from the assay substrate. Presence of the label on the assay substrate indicates the presence of the primary binding pair molecule in the biological sample.

The label may include a directly detectable label, which may be visible to a human observer, such as gold particles in a colloid or solution, commonly referred to as colloidal gold.

The label may include an indirect label, such an enzyme whereby the enzyme works on a substrate to produce a detectable reaction product. For example, an enzyme may attach to the primary binding pair molecule, and a substance that the enzyme converts to a detectable signal, such as a fluorescence signal, is contacted to the assay substrate. When light is directed at the assay substrate, any binding pair molecule complexes will fluoresce so that the presence of the primary binding pair molecule is observable.

An immunoassay may utilize one or more fluid solutions, which may include a dilutent solution to fluidize the biological sample, a conjugate solution having the labeled secondary binding pair molecules, and one or more wash solutions. The biological sample and fluids may be brought into contact, concurrently or sequentially with the assay substrate. The assay substrate may include an assay surface or an assay membrane, prepared with a coating of the corresponding binding pair molecules.

As described above, the second binding pair molecules may include an antigen that is specific to an antibody to be detected in a biological sample, or may include antibody that is specific to an antigen to be detected in the biological sample. By way of illustration, if the primary binding pair molecule to be detected is an antigen, the immobilized binding pair molecule and the secondary labeled binding pair molecule will be antibodies, both of which react with the antigen. When the antigen is present in the biological sample, the antigen will be immobilized by the immobilized antibody and labeled by the labeled secondary antibody, to form a sandwich-like construction, or complex.

It is known that non-specific or un-reacted components may be beneficially removed using wash solutions, often between processes and/or prior to a label detection process, in order to improve sensitivity and signal-to-noise ratios of the assay. Other permutations are possible as well. For example, a conjugate solution, such as a labeled secondary binding pair molecule solution may be mixed with or act as a sample dilutent to advantageously transport the biological sample to the assay substrate, to permit simultaneous binding of the primary binding pair molecule and the labeled secondary binding pair molecule to the immobilized binding pair molecule. Alternatively, or additionally, the sample dilutent may include one or more detergents and/or lysing agents to advantageously reduce deleterious effects of other components of the biological sample such as cellular membranes, non-useful cells like erythrocytes and the like.

Those skilled in the art will readily recognize that such fluid components and the order of the reactionary steps may be readily adjusted along with concentrations of the respective components in order to optimize detection or distinguishment of analytes, increase sensitivity, reduce non-specific reactions, and improve signal to noise ratios.

As will be readily understood, if the secondary antibody is labeled with an enzyme instead of a fluorescent or other immediately detectable label, an additional substrate may be utilized to allow the enzyme to produce a reaction product which will be advantageously detectable. An advantage of using an enzyme based label is that the detectable signal may increase over time as the enzyme works on an excess of substrate to produce a detectable product.

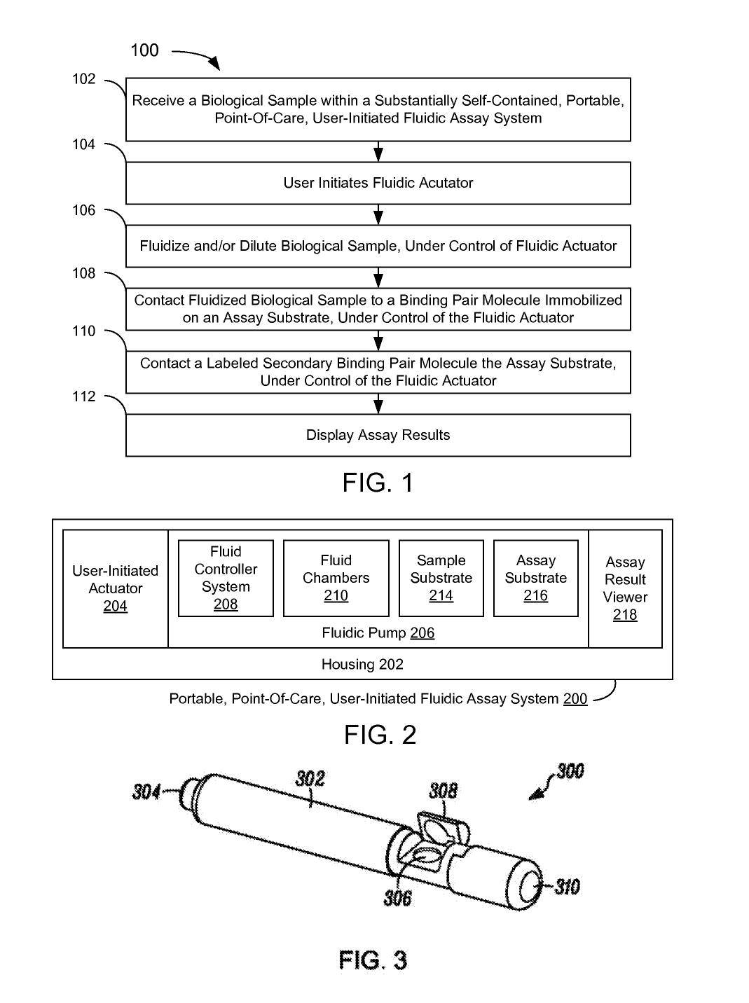

FIG. 1 is a process flowchart of a method 100 of detecting a primary binding pair molecule in a biological sample, using a substantially self-contained, point-of-care, user-initiated fluidic assay system. The primary binding pair molecule may correspond to an antibody or an antigen.

At 102, a biological sample is provided to the assay system. The biological sample may include one or more of a blood sample, a saliva sample, and a urine sample. The biological sample may be applied to a sample substrate within the assay system.

At 104, a fluidic actuator within the assay system is initiated by a user. The fluidic actuator may include a mechanical actuator, such as a compressed spring actuator, and may be initiated with a button, switch, or lever. The fluidic actuator may be configured to impart one or more of a physical force, pressure, centripetal force, gas pressure, gravitational force, and combinations thereof, on a fluid controller system within the assay system.

At 106, the biological sample is fluidized with a dilutent fluid. The dilutent fluid may flow over or through the sample substrate, under control of the fluid controller system.

At 108, the fluidized biological sample is contacted to a corresponding binding pair molecule that is specific to primary binding pair molecule. The corresponding binding pair molecule may be immobilized on an assay substrate within the assay system. The fluidized biological sample may flow over or through the assay substrate, under control of the fluid controller system.

Where the fluidized biological sample includes the primary binding pair molecule, the primary binding pair molecule attaches to the corresponding binding pair molecule and becomes immobilized on the assay substrate. For example, where the second binding pair molecule includes a portion of a pathogen, and where the biological sample includes an antibody to the pathogen, the antibody attaches to the antigen immobilized at the assay substrate.

At 110, a labeled conjugate solution is contacted to the assay substrate, under control of the fluid controller system. The labeled conjugate solution includes a secondary binding pair molecule to bind with the primary binding pair molecule. Where the primary binding pair molecule is immobilized on the assay substrate with the corresponding binding pair molecule, the secondary binding pair molecule attaches to the immobilized primary binding pair molecule, effectively creating a sandwich-like construct of the primary binding pair molecule, the corresponding binding pair molecule, and the labeled secondary binding pair molecule.

The secondary binding pair molecule may be selected as one that targets one or more proteins commonly found in the biological sample. For example, where the biological sample includes a human blood sample, the secondary binding pair molecule may include an antibody generated by a non-human animal in response to the one or more proteins commonly found in human blood.

The secondary binding pair molecule may be labeled with human-visible particles, such as a gold colloid, or suspension of gold particles in a fluid such as water. Alternatively, or additionally, the secondary binding pair molecule may be labeled with a fluorescent probe.

Where the labeled secondary binding pair molecule attaches to a primary binding pair molecule that is attached to a corresponding binding pair molecule, at 110, the label is viewable by the user at 112.

Method 100 may be implemented to perform multiple diagnostic assays in an assay system. For example, a plurality of antigens, each specific to a different antibody, may be immobilized on one or more assay substrates within an assay system. Similarly, a plurality of antibodies, each specific to a different antigen, may be immobilized on one or more assay substrates within an assay system

FIG. 2 is a block diagram of a portable, point-of-care, user-initiated fluidic assay system 200, including a housing 202, a user-initiated actuator 204, a fluidic pump 206, and an assay result viewer 218.

Pump 206 includes one or more fluid chambers 210, to contain fluids to be used in an assay. One or more of fluid chambers 210 may have, without limitation, a volume in a range of 0.5 to 2 milliliters.

Pump 206 includes a sample substrate 214 to hold a sample. Sample substrate 214 may include a surface or a membrane positioned within a cavity or a chamber of housing 202, to receive one or more samples, as described above.

Sample substrate 214 may include a porous and/or absorptive material, which may be configured to absorb a volume of liquid in a range of 10 to 500 μL, including within a range of up to 200 μL, and including a range of approximately 25 to 50 μL.

Pump 206 includes an assay substrate 216 to hold an assay material. Assay substrate 216 may include a surface or a membrane positioned within a cavity or chamber of housing 202, to receive one or more assay compounds or biological components, such as an antigen or an antibody, as described above.

Fluid chambers 210 may include a waste fluid chamber.

Pump 206 further includes a fluid controller system 208, which may include a plurality of fluid controllers, to control fluid flow from one or more fluid chambers 212 to one or more of sample substrate 214 and assay substrate 216, responsive to actuator 204.

Actuator 204 may include a mechanical actuator, which may include a compressed or compressible spring actuator, and may include a button, switch, lever, twist-activator, or other user-initiated feature.

Assay result viewer 218 may include a display window disposed over an opening through housing 202, over assay substrate 216.

FIG. 3 is a perspective view of a portable, point-of-care, user-initiated fluidic assay system 300, including a housing 302, a user-initiated actuator button 304, a sample substrate 306, and a sample substrate cover 308. Sample substrate cover 308 may be hingedly coupled to housing 302.

Assay system 300 further includes an assay result viewer 310, which may be disposed over an assay substrate. Assay result view 310 may be disposed at an end of assay system 300, as illustrated in FIG. 3, or along a side of assay system 300.

Assay system 300 may have, without limitation, a length in a range of 5 to 8 centimeters and a width of approximately 1 centimeter. Assay system 300 may have a substantially cylindrical shape, as illustrated in FIG. 3, or other shape.

Assay system 300, or portions thereof, may be implemented with one or more substantially rigid materials, and/or with one or more flexible or pliable materials, including, without limitation, polypropylene.

Example portable, point-of-care, user-initiated fluidic assay systems are disclosed further below.

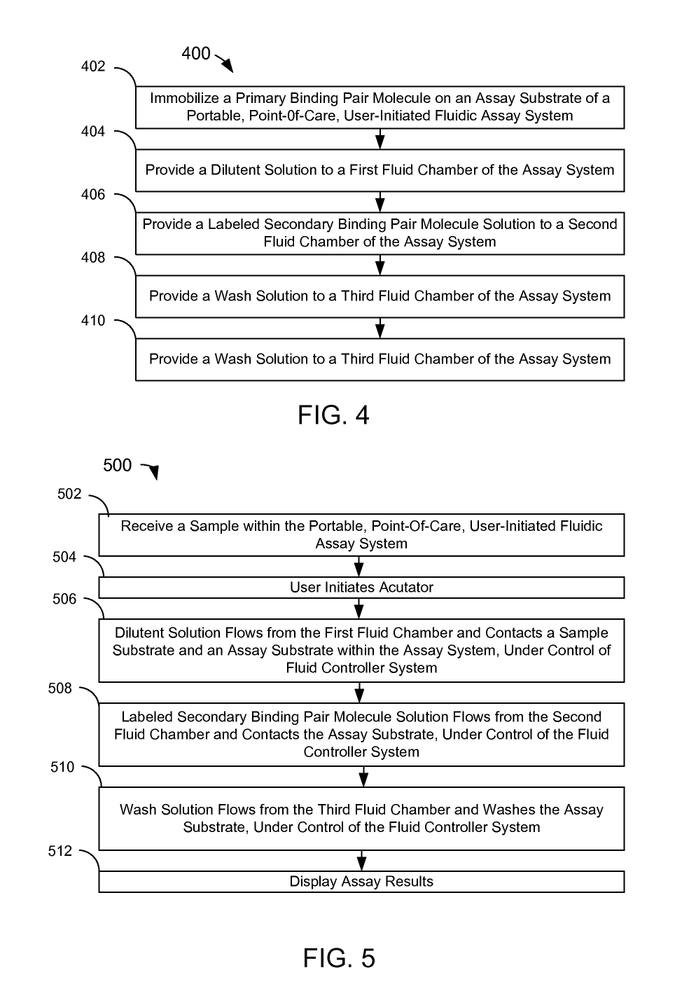

FIG. 4 is a process flowchart of a method 400 of preparing a portable, point-of-care, user-initiated fluidic assay system. Method 400 is described below with reference to assay system 200 in FIG. 2, for illustrative purposes. Method 400 is not, however, limited to the example of FIG. 2.

At 402, a binding pair molecule is immobilized on an assay substrate, such as assay substrate 216 in FIG. 2. The binding pair molecule may include an antigen specific to an antibody, or an antibody specific to an antigen.

At 404, a first one of fluid chambers 210 is provided with a dilutent solution to fluidize a sample.

At 406, a second one of fluid chambers 210 is provided with a labeled secondary binding pair molecule solution.

At 408, a third one of fluid chambers 210 is provided with a wash solution, which may include one or more of a saline solution and a detergent. The wash solution may be substantially similar to the dilutent solution.

FIG. 5 is a process flowchart of a method 500 of using an assay system prepared in accordance with method 400. Method 500 is described below with reference to assay system 200 in FIG. 2, and assay system 300 in FIG. 3, for illustrative purposes. Method 500 is not, however, limited to the examples of FIG. 2 and FIG. 3.

At 502, a sample is provided to a sample substrate, such as sample substrate 214 in FIG. 2, and sample substrate 306 in FIG. 3.

At 504, a user-initiated actuator is initiated by the user, such as user-initiated activator 204 in FIG. 2, and button 304 in FIG. 3. The user initiated actuator acts upon a fluid controller system, such as fluid controller system 208 in FIG. 2.

At 506, the dilutent solution flows from first fluid chamber and contacts the sample substrate and the assay substrate, under control of the fluid controller system.

As the dilutent fluid flows over or through the sample substrate, the sample is dislodged from the sample substrate and flows with the dilutent solution to the assay substrate.

At 508, the labeled secondary binding pair solution flows from the second fluid chamber and contacts the assay substrate, under control of the fluid controller system. The labeled secondary binding pair solution may flow directly to the assay substrate or may flow over or through the sample substrate.

At 510, the wash solution flows from the third fluid chamber and washes the assay substrate, under control of fluid controller system 208. The wash solution may flow from the assay substrate to a waste fluid chamber,

At 512, assay results are viewable, such as at assay result viewer 218 in FIG. 2, and assay result viewer 310 in FIG. 3.

An assay substrate may include a nitrocellulose-based membrane, available from Invitrogen Corporation, of Carlsbad, Calif.

Preparation of a nitrocellulose-based membrane may include incubation for approximately thirty (30) minutes in a solution of 0.2 mg/mL protein A, available from Sigma-Aldrich Corporation, of St. Louis, Mo., in a phosphate buffered saline solution (PBS), and then dried at approximately 37° for approximately fifteen (15) minutes. 1 μL of PBS may be added to the dry membrane and allowed to dry at room temperature. Alternatively, 1 μL of an N-Hydroxysuccinimide (NHS) solution, available from Sigma-Aldrich Corporation, of St. Louis, Mo., may be added to the dry membrane and allowed to dry at room temperature.

An assay method and/or system may utilize or include approximately 100 μL of PBS/0.05% Tween wash buffer, available from Sigma-Aldrich Corporation, of St. Louis, Mo., and may utilize or include approximately 100 μL of protein G colloidal gold, available from Pierce Corporation, of Rockland, Ill.

An assay method and/or system may be configured to test for Chlamydia, and may utilize or include a sample membrane treated with wheat germ agglutinin, to which an approximately 50 μL blood sample is applied. Approximately 150 μL of a lysing solution may then be passed through the sample membrane and then contacted to an assay substrate. Thereafter, approximately 100 μL of a colloidal gold solution may be contacted to the assay substrate. Thereafter, approximately 500 μL of a wash solution, which may include the lysing solution, may be contacted to the assay membrane without passing through the sample membrane.

Additional example assay features and embodiments are disclosed below. Based on the description herein, one skilled in the relevant art(s) will understand that features and embodiments described herein may be practiced in various combinations with one another.

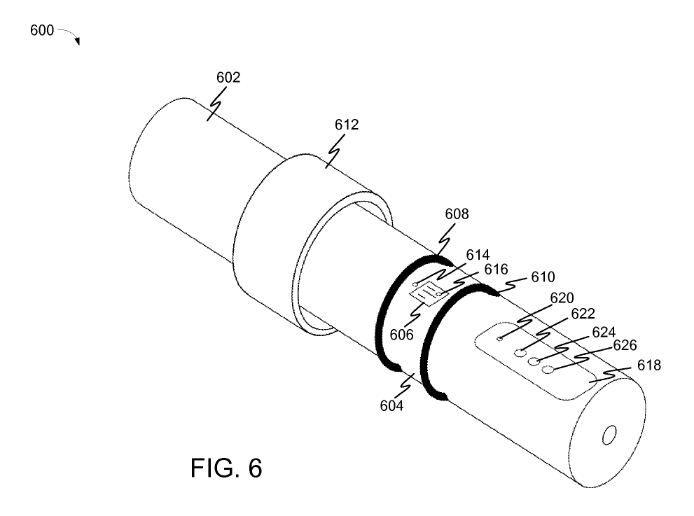

FIG. 6 is a perspective view of an assay system 600, including a body 602 having a sample collection region 604 to receive a sample collection pad or membrane 606, which may include a porous material such as, for example, a glass fiber pad, to absorb a fluid sample.

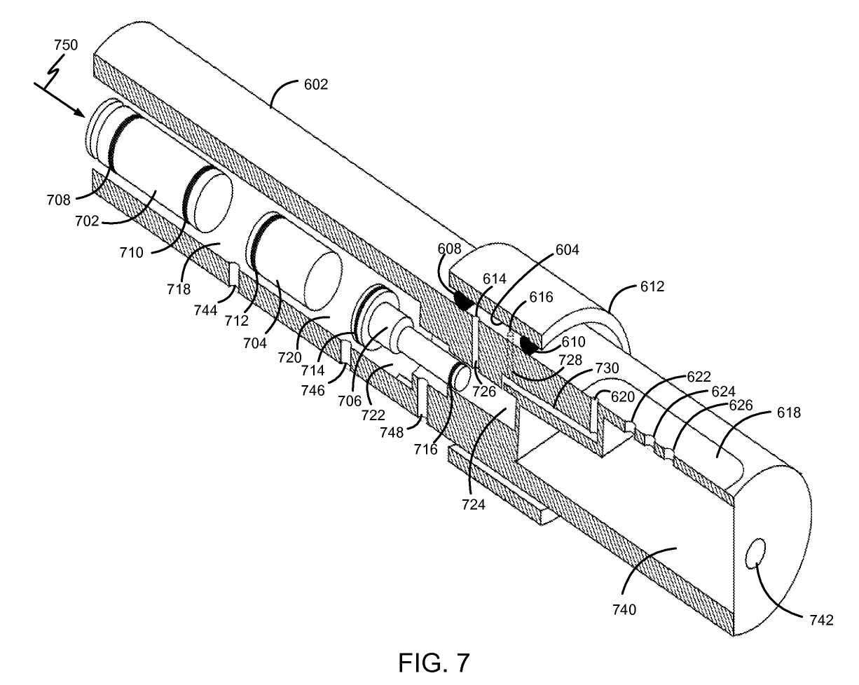

In the example of FIG. 6, sample collection region 604 is positioned between first and second O-rings 608 and 610, and system 600 includes a cover 612 slideably moveable relative to body 602, between a first position illustrated in FIG. 6, and a second position described below with reference to FIG. 7.

FIG. 7 is a cross-sectional view of assay system 600, wherein cover 612 is illustrated in the second position, and sample collection region 604 is bounded by an outer surface of body 602, an inner-surface of cover 612, and O-rings 608 and 610. O-rings 608 and 610 may provide a hermetic seal between sample collection region 604 and an external environment. When cover 612 is in the second position, sample collection region 604 may be referred to as a sample collection chamber.

In FIG. 6, sample collection region 604 includes openings 614 and 616 through the surface of body 602 associated with fluid passages within body 602. Opening 614 may be positioned adjacent to sample collection pad 606, and opening 616 may be positioned beneath sample collection pad 606. System 600 may be configured to provide a fluid through opening 614 into sample collection region 604 and to receive the fluid from sample collection region 604 through opening 616, to cause the fluid to pass through sample collection pad 606.

Body 602 may include an assay region 618 formed or etched within the surface of body 602, having an opening 620 through the surface of body 602 to receive fluid from an associated fluid passage. Assay region 618 may include one or more additional openings to corresponding fluid passages within body 602, illustrated here as openings 622, 624, and 626, to permit the fluid to exit assay region 618.

Assay region 618 may be configured to receive a test membrane having one or more reactive areas, each reactive area positioned on the test membrane in alignment with a corresponding one of openings 622, 624, and 626.

System 600 may include a substantially transparent cover to enclose assay region 618, such as to permit viewing of the test membrane, or portions thereof. The cover may include one or more fluid channels to direct fluid from opening 620 to the membrane areas aligned with openings 622, 624, and 626. Where system 600 includes a cover over assay region 618, assay region 618 may be referred to as an assay chamber.

In FIG. 7, system 600 includes plungers 702, 704, and 706. Plunger 706 is illustrated here as a multi-diameter or stepped plunger. Plunger 702 includes O-rings 708 and 710. Plunger 704 includes an O-ring 712. Plunger 706 includes O-rings 714 and 716. O-rings 708, 710, 712, 714, and 716 may be sized to engage corresponding inner surface portions of body 602. Plungers 702, 704, and 706 are each moveable within body 602 between respective first and second positions and, together with the inner surfaces of body 602, define fluid chambers 718, 720, 722, and 724.

In the example of FIG. 7, body 602 includes fluid passages 726 and 728 between corresponding openings 614 and 616 and fluid chamber 724, a fluid passage 730 between fluid chamber 724 and opening 620 of assay region 618, and fluid passages between each of openings 622, 624, and 626 of assay region 618 and a waste chamber 740. Waste chamber 740 may include an absorptive material to receive fluid from one or more fluid chambers of system 600. Body 602 may include a fluid passage 742 between waste chamber 740 and the outer surface of body 602, such as to release air displaced by fluid received within waste chamber 740.

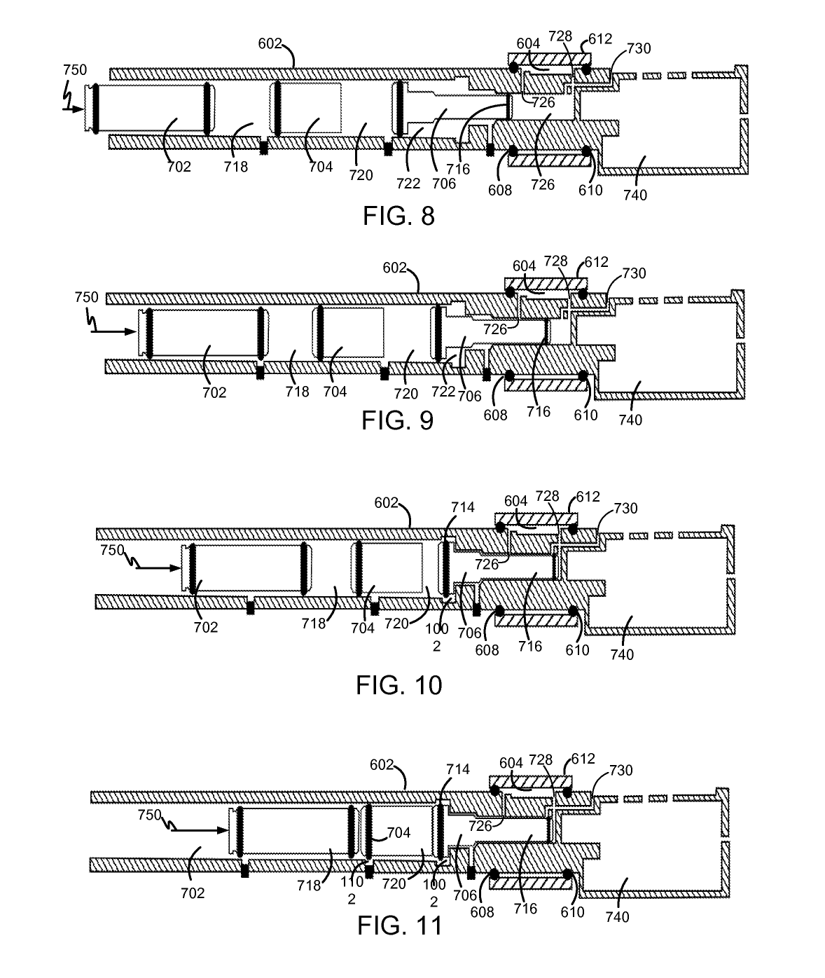

Body 602 may include one or more of fluid passages 744, 746, and 748 in fluid communication with corresponding fluid chambers 718, 720, and 722. One or more of fluid passages 744, 746, and 748 may have an opening through the outer surface of body 602, which may be used to provide one or more assay fluids to a corresponding fluid chamber during preparation procedure. Such an opening through the outer surface of body 602 may be plugged or sealed subsequent to the preparation procedure, such as illustrated in FIGS. 8-11. Alternatively, or additionally, one or more of fluid passages 744, 746, and 748 may include an opening to another fluid chamber of system 600, such as to provide a fluid bypass around one or more other fluid chambers and/or plungers.

Example operation of system 600 is described below with reference to FIGS. 8-14.

FIG. 8 is a cross-sectional view of system 600, wherein plungers 702, 704, and 706 are in corresponding initial or first positions.

FIG. 9 is a cross-sectional view of system 600, wherein plungers 702, 704, and 706 are in respective first intermediate positions.

FIG. 10 is a cross-sectional view of system 600, wherein plunger 704 is in a second position, and plungers 702 and 704 are in respective second intermediate positions.

FIG. 11 is a cross-sectional view of system 600, wherein plungers 702, 704 and 706 are in respective second positions.

FIGS. 8-11 may represent sequential positioning of plungers 702, 704 and 706 in response to a force in a direction 750 of FIG. 7.

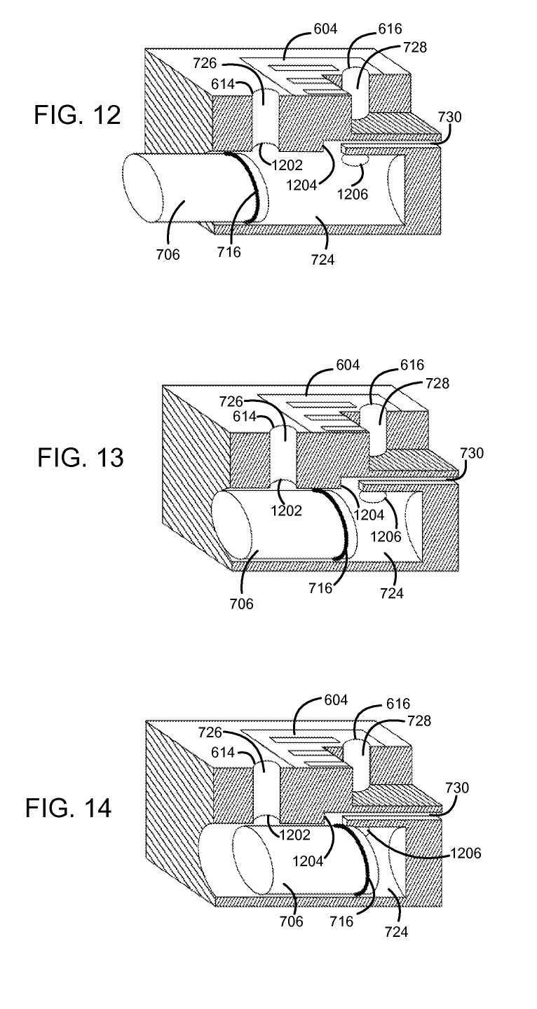

FIG. 12 is an expanded view of a portion of system 600, including a portion of plunger 706 in the first position corresponding to FIG. 8.

FIG. 13 is an expanded view of a of portion system 600, including a portion of plunger 706 in the intermediate position corresponding to FIG. 9, and including fluid directional arrows.

FIG. 14 is an expanded view of a portion of system 600, including a portion of plunger 706 in the second position corresponding to FIGS. 10 and 11.

During a preparation process, fluid chambers 718, 720, and 722, may be provided with corresponding first, second, and third fluids, and fluid chamber 724 may provided with a gas, such as air. The fluids in one or more of fluid chambers 718, 720, and 722 may be relatively incompressible compared with the gas in fluid chamber 724.

In FIG. 8, when the force is applied to plunger 702 in direction 750, the relatively incompressibility of the fluids in fluid chambers 718 and 720 transfer the force to plunger 706. Plungers 702, 704, and 706 may move together in direction 750.

As plungers 702, 704, and 706 move in direction 750, fluid within fluid chamber 724, which may include air, travels from fluid chamber 724, through fluid passage 730 to assay chamber 732, and through fluid passages 734, 736, and 738 to waste chamber 740.

Prior to O-ring 716 of plunger 706 passing an opening 1202 (FIG. 12) of fluid passage 726, fluid chamber 722 is substantially isolated and no fluid flows from fluid chamber 722 to fluid channel 728 or from fluid chamber 722 to fluid chamber 724.

As O-ring 716 of plunger 706 moves towards opening 1202, and as fluid chamber 722 is correspondingly moved in direction 750 into a narrower-diameter inner surface portion of body 602, a volume of fluid chamber 722 decreases. The reduced volume of fluid chamber 722 may increase a pressure of the fluid within fluid chamber 722. The fluid within fluid chamber 722 may include a combination of a relatively incompressible fluid and relatively compressible fluid, such as air, which may compress in response to the increased pressure.

In FIG. 9, when O-ring 716 is positioned between opening 1202 of fluid passage 726 and an opening 1204 of fluid passage 730, fluid chamber 722 is in fluid communication with fluid channel 726, while O-ring 716 precludes fluid flow directly between fluid chambers 722 and 724. The fluid in fluid chamber 722 may thus travel from fluid chamber 722, through fluid passage 726 to sample collection region 604, through fluid passage 728 to fluid chamber 724, through fluid passage fluid passage 730 to assay region 618, and through openings 722, 724, and 726 to waste chamber 740.

The fluid from fluid chamber 722 may contact and dislodge at least a portion of a sample contained within a sample pad 606, and may carry the sample to assay region 618, where the sample may react with a test membrane.

In FIG. 10, as plunger 706 reaches the second position and O-ring 716 passes opening 1204, a recess 1002 within an inner surface of body 602 provides a fluid passage around O-ring 714. Fluid within fluid chamber 720 travels through recess 1002, alongside plunger 706, through fluid passage 730 to assay chamber 732, and through fluid passages 734, 736, and 738 to waste chamber 740.

In FIG. 11, as plunger 704 reaches the second position, a recess 1102 within an inner surface of body 602 provides a fluid passage around O-ring 712 of plunger 704. Recess 1102 may correspond to fluid channel 746 in FIG. 7. Fluid within fluid chamber 718 travels through recess 1102, alongside plunger 704, through recess 102, alongside plunger 706, through fluid passage 730 to assay chamber 732, and through fluid passages 734, 736, and 738 to waste chamber 740.

As illustrated in FIG. 14, when plunger 706 is in the second position, O-ring 716 may be positioned between an opening 1402 of fluid channel 728 and an opening 1404 of fluid channel 730 to preclude fluid flow from sample collection region 604 to assay chamber 732 through fluid channels 728 and 730. This may be useful, for example, where the fluids within fluid chamber 720 and 718 are to contact an assay membrane within assay chamber 732 rather than sample pad 606 within sample collection region 604. This may be useful, for example, where the fluids within fluid chamber 720 and 718 include a wash fluid and/or a reactive material to wash and/or react with the assay membrane.

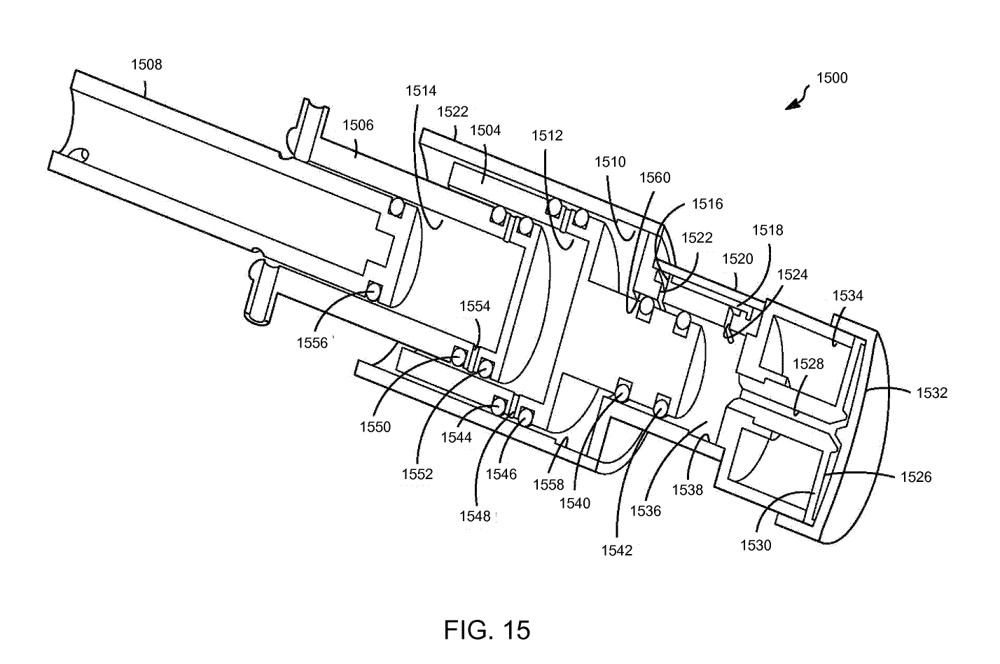

FIG. 15 is a cross-sectional perspective view of a portion of an assay system 1500 including a housing portion 1502 and a fluid controller system, including a plurality of fluid controllers, or plungers 1504, 1506, and 1508. Fluid controllers 1504, 1506, and 1508 define a plurality of fluid chambers, illustrated here as first, second, and third fluid chambers 1510, 1512, and 1514, respectively. Fluid controllers 1504, 1506, and 1508 are slideably nested within one another.

Housing portion 1502 includes a sample chamber 1516 to receive a sample, and may include a sample substrate, membrane or pad 1518. Housing portion 1502 may include a cover mechanism such as a cover portion 1520, which may be removable or hingedly coupled to housing portion 1502, as described above with respect to FIG. 3. Housing portion 1502 includes a sample chamber inlet 1522 and a sample chamber outlet 1524.

Housing portion 1502 includes an assay chamber 1526 and an assay chamber inlet 1528, and may include an assay substrate, membrane or pad 1528 to capture, react, and/or display assay results.

Housing portion 1502 includes an assay result viewer, illustrated here as a display window 1532 disposed over assay chamber 1528.

Housing portion 1502 includes a waste fluid chamber 1534 to receive fluids from assay chamber 1526.

Housing portion 1502 includes a transient fluid chamber 1536 having one or more fluid channels 1538, also referred to herein as a fluid controller bypass channel.

Housing portion 1502 further includes one or more other fluid channels 1558.

First fluid chamber 1510 includes a fluid chamber outlet 1560, illustrated here as a space between fluid controller 1506 and an inner surface of hosing portion 1502.

Second fluid chamber 1512 includes a fluid chamber outlet 1548, illustrated here as a gate or passage through fluid controller 1504.

Third fluid chamber 1514 includes a fluid chamber outlet 1554, illustrated here as a gate through fluid controller 1506.

Fluid controllers 1504, 1506, and 1508 include one or more sealing mechanisms, illustrated here as O-rings 1540 and 1542, O-rings 1544 and 1546, O-rings 1550 and 1552, and O-ring 1556.

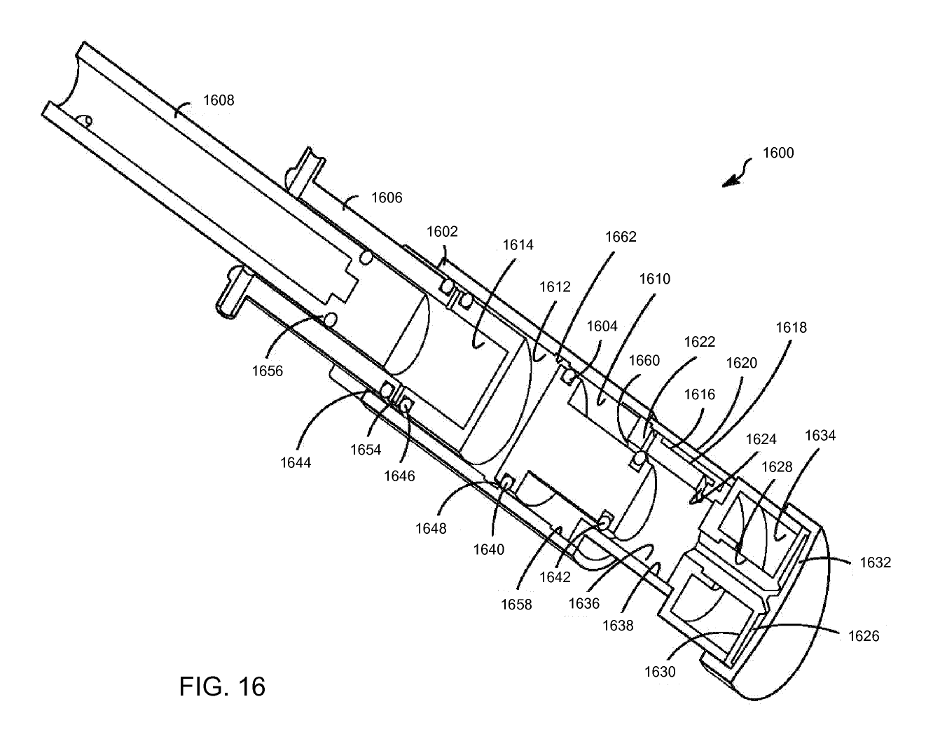

FIG. 16 is a cross-sectional perspective view of a portion of an assay system 1600 including a housing portion 1602 and a fluid controller system, including a plurality of fluid controllers, or plungers 1604, 1606, and 1608. Fluid controllers 1604, 1606, and 1608 define a plurality of fluid chambers, illustrated here as first, second, and third fluid chambers 1610, 1612, and 1614, respectively. Fluid controller 1608 is slideably nested within fluid controller 1606.

Housing portion 1602 includes a sample chamber 1616 to receive a sample, and may include a sample substrate 1618, which may include a surface of sample chamber 1616 or membrane therein. Housing portion 1602 may include a cover mechanism such as a cover portion 1620, which may be removable or hingedly coupled to housing portion 1602, as described above with respect to FIG. 3. Housing portion 1602 includes a sample chamber inlet 1622 and a sample chamber outlet 1624.

Housing portion 1602 includes an assay chamber 1626 and an assay chamber inlet 1628, and may include an assay substrate 1628 to capture, react, and/or display assay results. Assay substrate may include a surface of assay chamber 1626 or a membrane therein.

Housing portion 1602 includes an assay result viewer, illustrated here as a display window 1632 disposed over assay chamber 1628.

Housing portion 1602 includes a waste fluid chamber 1634 to receive fluids from assay chamber 1626.

Housing portion 1602 includes a transient fluid chamber 1636 having one or more fluid channels 1638, also referred to herein as a fluid controller bypass channel.

Housing portion 1602 further includes fluid channels 1658 and 1662.

First fluid chamber 1610 includes a fluid chamber outlet 1660, illustrated here as a space between fluid controller 1606 and an inner surface of hosing portion 1602.

Second fluid chamber 1612 includes a fluid chamber outlet 1648, illustrated here as a space between fluid controller 1604 and an inner surface of hosing portion 1602.

Third fluid chamber 1614 includes a fluid chamber outlet 1654, illustrated here as a gate or passage through fluid controller 1606.

Fluid controllers 1604, 1606, and 1608 include one or more sealing mechanisms, illustrated here as O-rings 1640 and 1642, O-rings 1644 and 1646, and O-ring 1656.

One or more inlets, outlets, openings, channels, and fluid pathways as described herein may be implemented as one or more of gates and passageways as described in one or more preceding examples, an may include one or more of:
a fluid channel within an inner surface of a housing;
a fluid passage within a housing, having a plurality of openings through an inner surface of the housing;
the fluid passage through a fluid controller; and
a fluid channel formed within an outer surface of one of the fluid controllers.

One or more inlets, outlets, openings, channels, fluid paths, gates, and passageways, as described herein, may include one or more flow restrictors, such as check valves, which may include a frangible check valve, to inhibit fluid flow when a pressure difference across the flow restrictor valve is below a threshold.

In FIG. 2, user-initiated actuator 204 may include one or more of a mechanical actuator, an electrical actuator, an electro-mechanical actuator, and a chemical reaction initiated actuator. User-initiated actuator systems are disclosed below, one or more of which may be implemented with pumps disclosed above.

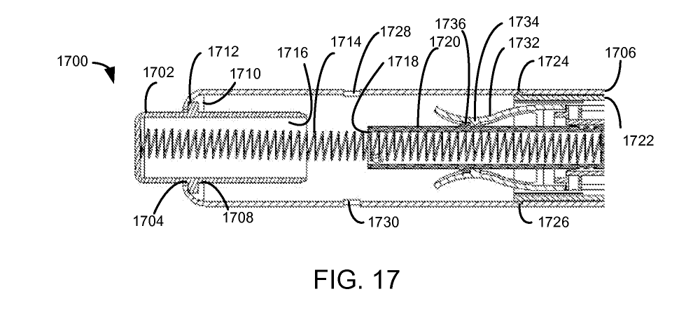

FIG. 17 is cross-sectional view of a mechanical actuator system 1700. Actuator system 1700 includes a button 1702 slideably disposed through an opening 1704 of an outer housing portion 1706, and through an opening 1708 of a frangible inner wall 1710 of outer housing portion 1706. Button 1702 includes a detent 1712 that extends beyond openings 1704 and 1708 to secure button 1702 between housing portion 1706 and frangible inner wall 1710.

Actuator system 1700 includes a compressible spring 1714 having a first end positioned within a cavity 1716 of button 1702, and a second end disposed within a cavity 1718 of a member 1720. Member 1720 may be coupled to, or may be a part of a fluid controller system, such a part of a plunger or fluid controller as described and illustrated in one or more examples herein.

Actuator system 1700 includes an inner housing portion 1722, slideably engaged within outer housing portion 1706. Inner housing portion 1722 includes one or more detents, illustrated here as detents 1724 and 1726, to lockingly engage one or more corresponding openings 1728 and 1730 in an inner surface of outer housing portion 1702.

Actuator system 1700 includes one or more frangible snaps 1732 coupled, directly or indirectly, to inner housing portion 1722. Frangible snap 1732 includes a locking detent 1734, and member 1720 includes a corresponding locking detent 1736 to releasably couple member 1720 to frangible snap 1732.

An assay system as disclosed herein may include a user-rupturable membrane to separate a plurality of chemicals within a flexible tear-resistant membrane. The chemicals may be selected such that, when combined, a pressurized fluid is generated. The pressurized fluid may be gas or liquid. The pressurized fluid may cause fluid controllers to move as described in one or more examples above. Multiple user-rupturable membranes may be implemented for multiple fluid passages.

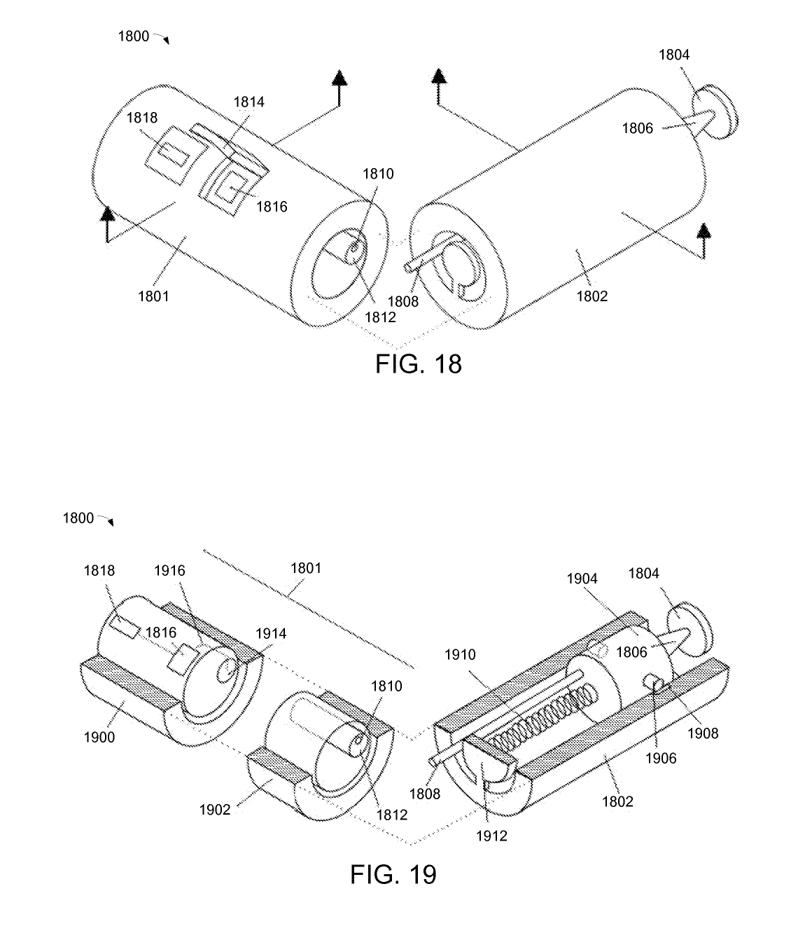

Additional example methods and systems to activate assay systems are disclosed below with respect to FIGS. 18-23.

FIG. 18 is a profile view of another fluidic assay system 1800 including first and second portions or sections 1801 and 1802.

FIG. 19 is a cross-sectional view of system 1800, including elements 1900 and 1902 of portion 1801.

Section 1802 may include a user initiated thumb press, pushbutton, or cap 1804 that is connected to a rotational post, rod or axle 1806 that is affixed to a control device 1904 which may have directional control post 1906 that is contained in an internal track 1908. Rotational post 1806 allows control device 1904 to rotate independently of the thumb press 1804.

Section 1902 may include one or more fluid chambers, cavities, and/or voids 1812 that are activated by the rod 1808 connected to the control device 1904. The fluid chambers may contain plungers separating fluid, fluid under pressure, or fluid packages that burst when activated at point 1810 by rod 1808.

Section 1801 may contain fluid pathways, channels, or ducts 1916 that connect to one or more of a sample collection region 1816 and a sample analyzing or assay region 1818, which may include an assay observation region. Sample collection region 1816 may have a glass fiber pad or other material that can absorb a sample and a cover or window 1814 to seal the chamber to prevent additional sample from being applied.

Sample analyzing region 1818 may have a test membrane, such as nitrous cellulose, with immobilized antigens or other substance to detect the presence of certain analytes in the sample.

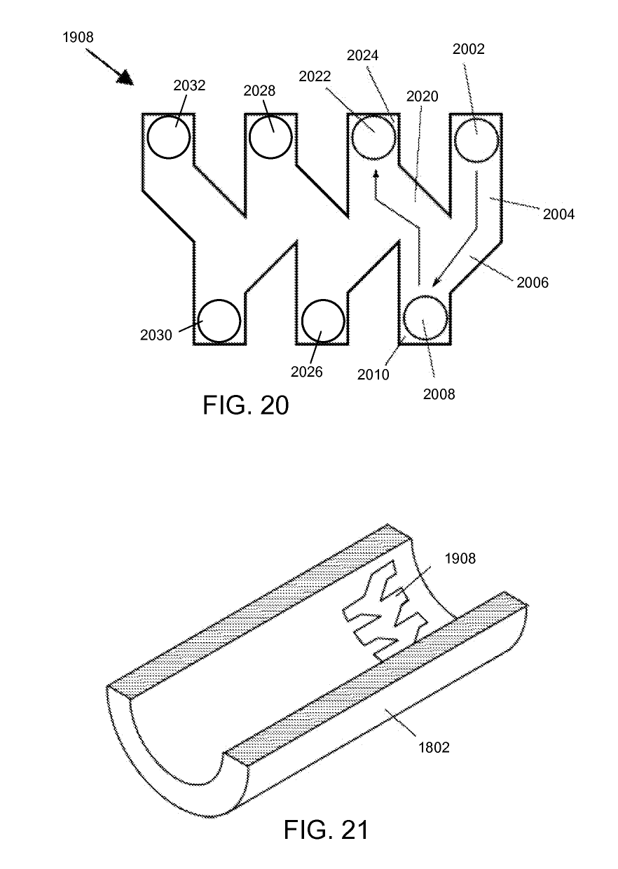

Example operation of control post 1906 is described below with reference to FIGS. 20 and 21. FIG. 20 is a graphic depiction of an example pathway of internal track 1908 to guide control post 1906. FIG. 21 is corresponding a cross-sectional view of portion 1802, including an internal track 1908 formed within an inner surface thereof, corresponding to the pathway of FIG. 20. Internal track 1908 is not, however, limited to the examples of FIGS. 20 and 21.

In FIG. 20, control post 1906 slidingly engages track 1908 beginning at a position 2002, such as in response to a force applied to thumb press 1804 in FIG. 18, and in response to a force applied by spring 1910.

In response to a force applied to thumb press 1804 in a direction 2050, control post 2806 travels from position 2002, along a linear portion 2004 of track 1908, through an angled portion 2006, and into a position 2008. Position 2008 may correspond to a depressed position of thumb press 1804 and a corresponding compressed position of spring 1910.

As control post 1906 travels from position 2002 to position 2008, control device 1904 moves linearly relative to portion 1802, in direction 2050.

As control post 1906 travels along angled portion 2006, a directional or rotational force is imparted to control post 1906 by a side wall of angled portion 2006, to cause control device 1904 to rotate relative to portion 1802. A rotational distance of control unit 1904 may correspond to a shortest distance between portions 2006 and 2010.

Upon release of thumb press 1804, compressed spring 1910 applies a force to control post 1906 in a direction 2052, to cause control post 1906 to travel into an angled portion 2020 of path 1902, which guides control post 1906 into a position 2022 of a portion 2024.

As control post 1906 travels from position 2008 to position 2022, control device 1904 moves linearly relative to portion 1802, in direction 2052.

As control post 1906 travels along angled portion 2020, a directional or rotational force is imparted to control post 1906, such as by a side wall of angled portion 2020, to cause control device 1904 to further rotate relative to portion 1802. A rotational distance of control unit 1904 may correspond to a shortest distance between portions 2010 and 2024.

Thumb press 1804 may be repeatedly depressed and released to move control post 1906 along additional portions of track 1908, such as to positions 2026 through 2032.

Linear and/or rotation movement of control device 1904 relative to portion 1802 may control and/or align elements of system 1800 to activate and/or control one or more assay features. For example, linear and rotational movement of control device 1904 may position control rod 1808 in FIGS. 18 and 19 relative to one or more activation points associated with portion 1801.

For example, control rod 1808 may control movement of a plunger and/or other elements within portion 1801, relative to one or more other elements within portion 1801. Alternatively, or additionally, rod 1808 may include a fluid passage to permit fluid to flow between fluid chamber 1812 and another fluid chamber and/or fluid passage via rod 1808.

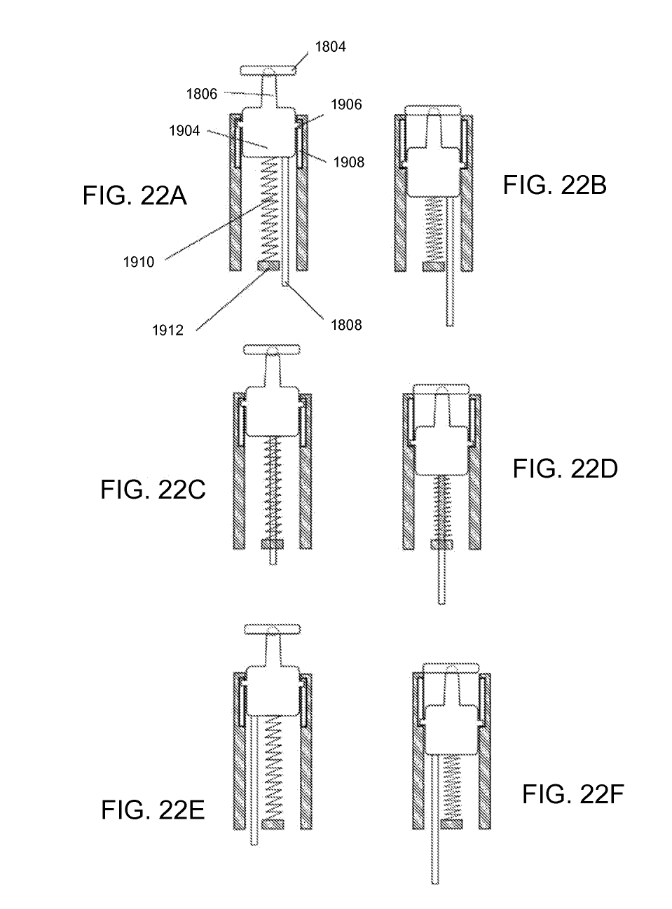

FIGS. 22A through 22F are cross-sectional views of portion 1802, depicting an example sequence of movements of in response to repeated pressing and releasing of thumb press 104. FIGS. 22A through 22F may correspond to positions 2002, 2008, 2022, 2026, 2028, and 2030, respectively, in FIG. 20. Each of the positions associated with FIGS. 22B through 22F may be associated with a corresponding assay activity. Alternatively, one or more of the positions associated with FIGS. 22B through 22F may correspond to an interim or transitory position, such as to provide a reaction time.

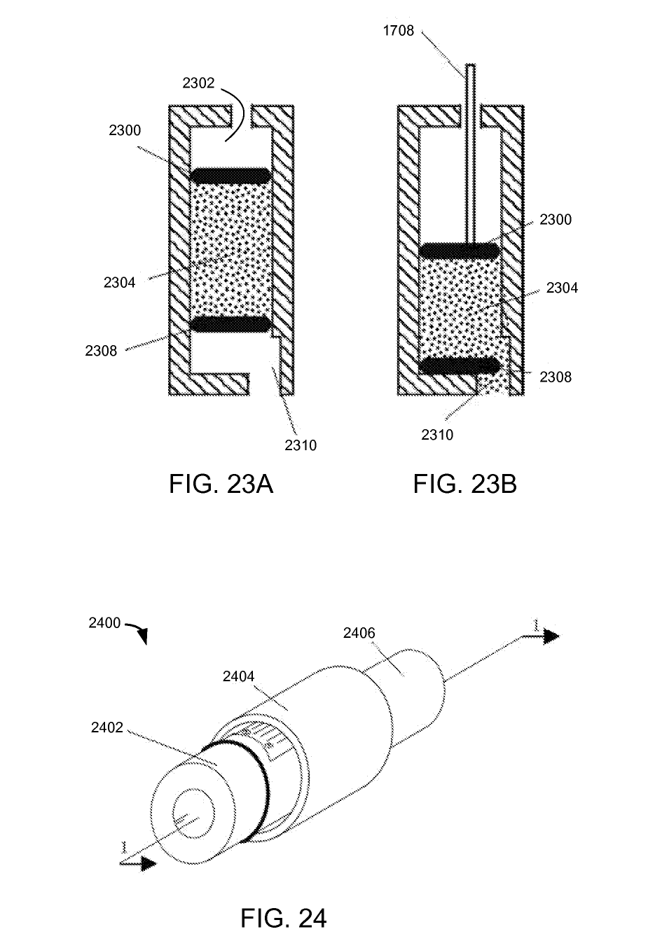

In FIG. 19, section 1902 may include one or more fluid chambers 1812 that are activated by the rod 1808 at point 1810. FIGS. 23A and 23B are cross-sectional views of a fluid chamber 2302, including first and second plungers 2300 and 2308.

In FIG. 23A, rod 1808 pushes down on first plunger 2300, which transfers force through fluid 2304 to second plunger 2308.

In FIG. 23B, the force applied to second plunger 2308 causes second plunger 2308 to move to an open position defined with respect to a bypass channel 2310, to release at least a portion of fluid 2304 from fluid chamber 2302.

Fluid exiting fluid chamber 2302 may enter a region 1914 in FIG. 19, which may be aligned with a fluid passage 1916. One or more other fluid chambers may also include fluid exit passages aligned with corresponding fluid passages 1916. One or more of the fluid passages 1916 may connect to sample collection region 1816 and/or to an assay reaction region 1818. Fluid channels that connect to the sample collection region 1816 may bring the sample to the assay reaction region 1818.

An assay apparatus as disclosed herein may be implemented to include all or substantially all components needed to carry out a test, such as an immunoassay, which may provide portability and which may permit operation relatively little training.

An assay apparatus as disclosed herein may be implemented to activate fluid chambers in a specific order, which may reduce the possibility of operator error.

An assay apparatus as disclosed herein may be implemented to provide a reaction time between pressings and/or releasing of thumb press 104. Such reaction time(s) may be controlled by a timer device and/or by an operator.

An assay apparatus as disclosed herein may be reconfigurable for different fluid components and/or assays. For example, a plurality of portions 1801 may each be configured differently from one another, and portion 1802 may be configured to couple to each of the plurality of portions 1801.

Portions of an assay apparatus as disclosed herein may be re-usable. For example, the sample collection and test section may be replaceable and disposable.

Methods and systems to collect samples are described below with respect to FIGS. 24-27.

FIG. 24 is a perspective view of a sample collection system 2400, including a body 2402, a cover 2404, and a plunger 2406.

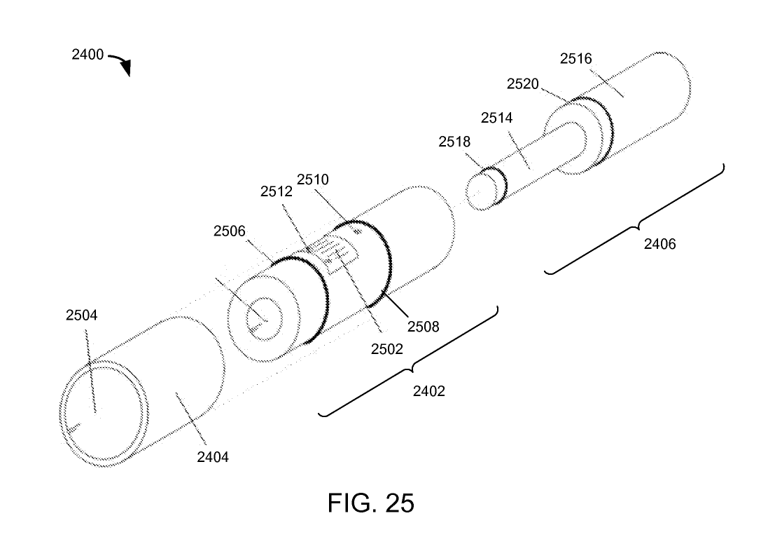

FIG. 25 is another perspective view of sample collection system 2400, wherein body 2402 includes a sample collection region 2502, and cover 2404 includes an inner surface 2504 sized to slideably enclose sample collection region 2502.

Sample collection system 2400 may include O-rings 2506 and 2508 to sealingly enclose or isolate sample collection region 2502 from an exterior environment.

Sample collection system 2400 is not, however, limited to a sliding cover. For example, sample collection system 2400 may include hinged cover, a rotating cover, a moveable or removable window, and/or other types of covers.

Sample collection region 2502 may be configured to receive a sample collection pad, which may include a glass fiber pad or other material to absorb an amount of sample. The sample collection pad may be sized to hold or absorb a relatively small amount of sample, such as, for example approximately 250 micro-liters (μl) or less, which is approximately a volume of a drop of blood from a finger puncture.

Alternatively, or additionally, sample collection region 2502 may include a non-absorbent structure, which may include micro-needles and/or other lance device, to capture a drop of blood, and/or a scraping device across which a swab map be provided to transfer a sample from the swab to the sample collection region.

Sample collection region 2502 may include one or more fluid entry lumens 2510 and one or more fluid exit lumens 2512.

In FIG. 25, plunger 2406 is illustrated as a stepped plunger having a plurality of regions of differing diameters or circumferences, including a first circumference region 2514 and a second circumference region 2516 that is greater than first circumference region 2514. In inner surface of body 2402 may be sized to accommodate circumference regions 2514 and 2516, and to define one or more fluid chambers. Sample collection system 2400 may include O-rings 2518 and 2520 to seal the fluid chambers.

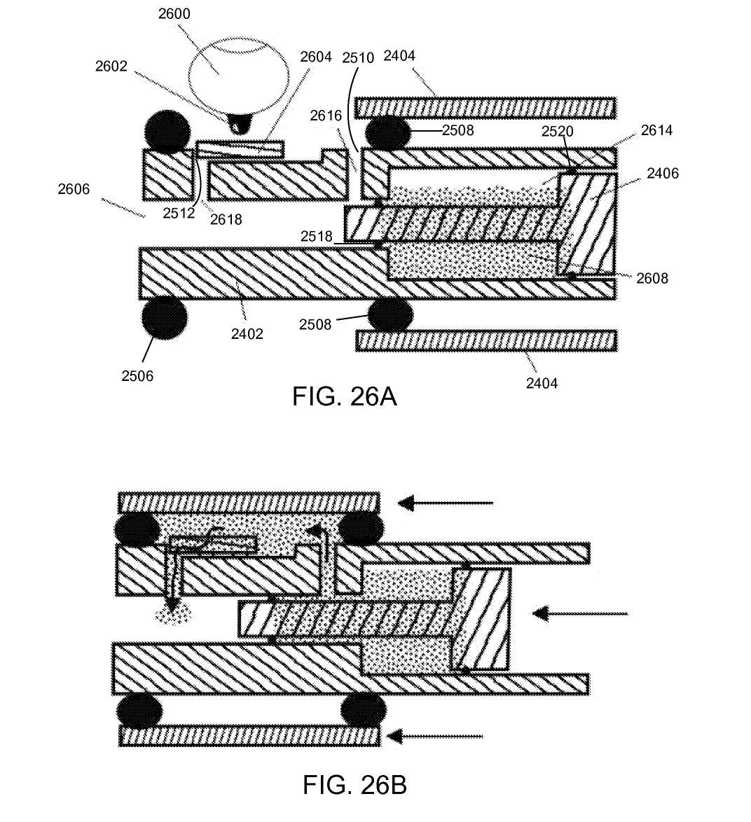

An example embodiment and corresponding operation of sample collection system 2400 is described below with respect to FIGS. 26A and 26B. FIG. 26A is a cross-sectional view of sample collection system 2400, wherein plunger 2406 is illustrated in a first position. FIG. 26B is another cross-sectional view of sample collection system 2400, wherein plunger 2406 is illustrated in a second position. In FIGS. 26A and 26B, a fluid chamber 2614 is defined by plunger 2406, an inner surface of body 2402, and O-rings 2610 and 2612. A volume of fluid chamber 2614 varies in response to the position of plunger 2406.

In FIG. 26A, a finger 2600 is lanced to expel a drop of blood 2602 to a blood collection pad 2604 within sample collection region 2502 of FIG. 25. Finger 2600 is subsequently withdrawn from sample collection region 2502 and cover 2404 is slid over sample collection region 2502.

In FIG. 26B, plunger 2406 is pushed towards a fluid passage 2606. Fluid 2608 within fluid chamber 2614 is forced into a fluid passage 2616 to entry lumen 2510. The fluid flows through sample collection pad 2604, where it mixes with or dislodges blood from sample 2602. Resultant blood containing fluid exits sample collection region 2502 through exit lumen 2512, and passes through a fluid passage 2618 to fluid passage 2606, from where it can exit the sample collection system 2400, such as for assay and/or storage.

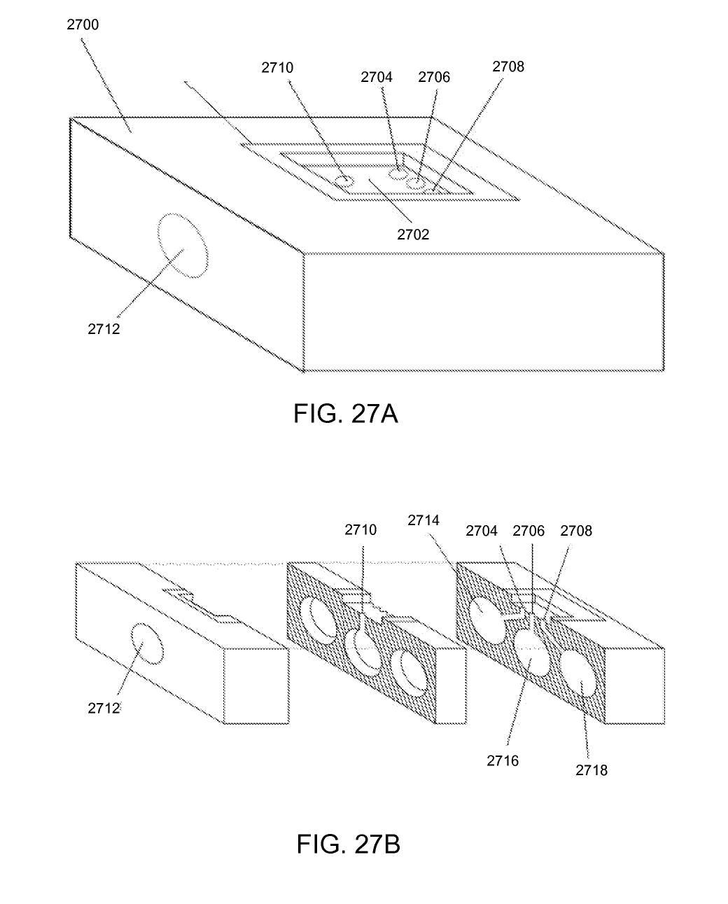

Another example embodiment of a sample collection system is described below with respect to FIGS. 27A and 27B.

FIG. 27A is a perspective view of a sample collection system 2700, including a sample chamber 2702 to receive fluids from multiple input lumens or fluid passages, illustrated here as fluid passages 2704, 2706, and 2708.

FIG. 27B is a cross-sectional view of sample collection system 2700, including fluid chambers 2214, 2716, and 2718, corresponding to fluid passages 2704, 2706, and 2708.

Sample collection system 2700 may correspond to a portion of body 2402 in FIG. 2 that includes sample collection region 2502.

Fluid passages 2704, 2706, and 2708 may each be controlled by a corresponding one of multiple plungers, which may be operated simultaneously, serially, and combinations thereof. The plungers may be configured as stepped plungers, such as described in one or more examples above. Sample collection system 2700 may include one or more passages to release air from one or more fluid chambers associated with one or more of the multiple plungers.

Sample collection system 2700 may include an exit lumen or fluid passage 2710 to allow fluid to exit sample chamber 2702 to an exit chamber 2712.

Sample collection system 2700 may operate, with respect to each of the multiple plungers, similarly to the description above with respect to FIGS. 26A and 26B.

Sample collection, as disclosed herein, may be implemented to fluidize, flush, flow, force, and/or dilute a sample, to provide at least a portion of the sample through a sample chamber fluid outlet.

A sample may include a biological sample, which may include, without limitation, blood, saliva, mucous, urine, feces, skin, and/or other biological substance(s), and may be obtained through one of more of puncture or piercing, swabbing, scraping, and excretion.

Sample collection methods and systems as disclosed herein may be implemented to receive a relatively small sample, such as a drop of blood, and to prepare the sample with one or more fluids before being applied to a test, such as an immunoassay.

Sample collection methods and systems as disclosed herein may be implemented to apply relatively significantly more fluid than contained in a sample, such as a lateral flow strip, that relies on the capillary action of fluid to carry out the test.

Sample collection methods and systems as disclosed herein may be implemented to receive a relatively small sample, and to prepare, store and/or ship the prepared sample to a lab, which may otherwise require significantly more sample to compensate for the loss of sample during transportation.

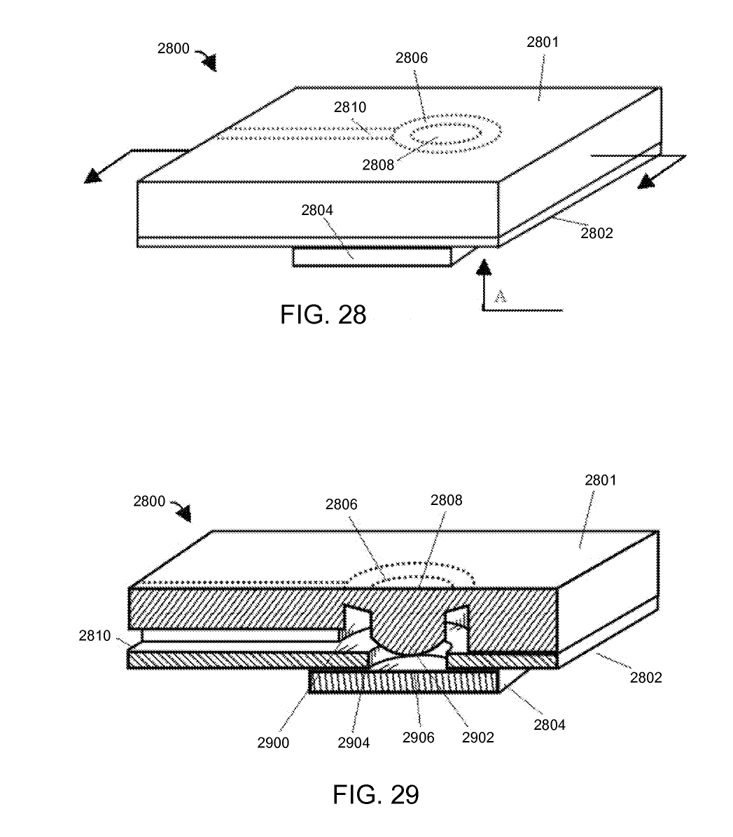

Methods and systems to trap or capture bubbles are disclosed below with respect to FIGS. 28-33.

FIG. 28 is a profile view of a bubble trap system 2800.

FIG. 29 is a cross-sectional view of bubble trap system 2800.

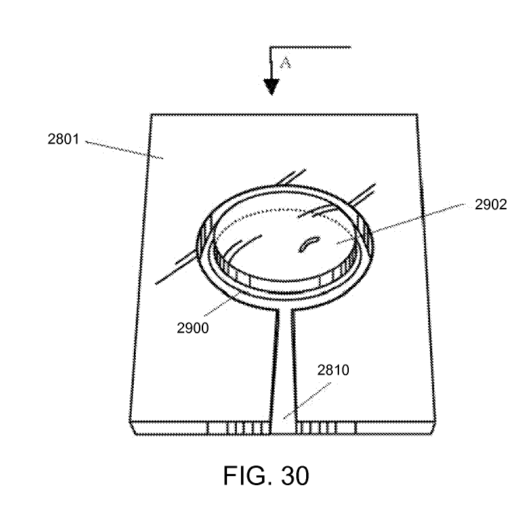

FIG. 30 is an upwardly directed view of an upper portion 2801 of bubble trap system 2800.

In FIG. 29, system 2800 includes a fluid channel 2810 to provide fluid to an opening or orifice 2904 through a surface of system 2800.

System 2800 may include a porous membrane 2804 positioned over orifice 2904 to receive fluid from fluid channel 2810. Porous membrane 2804 may include an active region, which may coincide with orifice 2904, and which may include a substance immobilized thereon. The substance may include, for example, an element to participate in a binding reaction, such as to detect the presence of a binding partner in a fluid sample.

System 2800 further includes a bubble termination pathway 2806 to receive, capture, or trap gas bubbles from fluid that flows through fluid channel 2810 to orifice 2904. Bubble termination pathway 2806, or a portion thereof, may be located vertically higher that at least a portion of fluid channel 2810 to permit gas bubbles to rise upwardly from fluid channel 2810. Gas bubbles may remain within bubble termination pathway 2806 due to buoyancy.

Bubble termination pathway 2806 may include a cavity 2900 (FIG. 29), having dimensions to hold a predetermined amount or volume of gas bubbles.

System 2800 may include a core portion 2808 having a lower surface 2902 disposed above orifice 2904 and defining a cavity 2906 therebetween. Lower surface 2902 may be substantially convex, which may assist in directing gas bubbles from cavity 2906, orifice 2904, and/or porous membrane 2804, toward cavity 2900, such as in response to gravity and/or centrifugal force.

Bubble termination pathway 2806, cavity 2900, core 2808, orifice 2904, and/or cavity 2906 may be in substantially vertical alignment with one another. Bubble termination pathway 2806, cavity 2900, core 2808, orifice 2904, and/or cavity 2906 may have substantially annular shapes, and may be in annular alignment with one another. Cavity 2900 may substantially encircle core 2808.

Bubble termination pathway 2806 may include a slanted upper surface, which may encourage distribution of gas bubbles throughout bubble termination pathway 2806.

Bubble termination pathway 2806, or a portion thereof, may be positioned outside of a circumference of orifice 2904, which may provide improved separation of gas bubbles from fluid, and which may provide an increased volume of space to hold or trap gas bubbles. permit increased.

Fluid channel 2810 may be in substantially horizontal alignment with a surface of core portion 2808, which may assist in separating gas bubbles from fluid, and which may assist in trapping gas bubbles in bubble termination pathway 2806.

System 2800 may include an upper portion 2801 and a lower portion 2802, which may be sealed together such as by adhesion, chemical solvents, or mechanical force (such as ultrasonics).

Upper portion 2801, or portions thereof, may be implemented with, for example, a substantially rigid clear material, such as a plastic, which may include one or more of styrene, polystyrene, nylon, polycarbonate or other suitable material.

Lower portion 2802, or portions thereof, may be implemented with, for example, a relative thin polystyrene material.

Porous membrane 2804 may be implemented with, for example, a nitrous cellulose membrane, and lower portion 2802 may be implemented with a material that can seal to a nitrous cellulose membrane 2804.

Bubble termination pathway 2806 and/or cavity 2900 may be sized to accommodate a predetermined, expected, or anticipated amount of gas to be trapped.

Orifice 2904 and/or an active area of porous membrane 2804 may be sized to expose a desired amount of membrane 2804 to accommodate the surface area of the active region to be in contact with a fluid. Orifice 2904 and/or an active area of porous membrane 2804 have a diameter of, for example, approximately 0.125 inches, which may provide for suitable involvement with the active region of the membrane although it will be readily recognized by those skilled in the art that many dimensions may be suitable depending on the assay to be performed, the strength of the detectable signal desired and the sensitivity to be achieved.

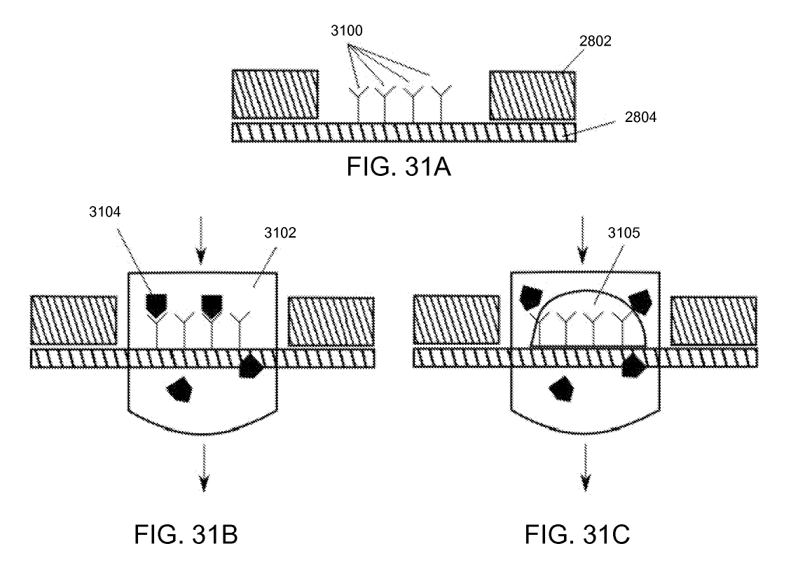

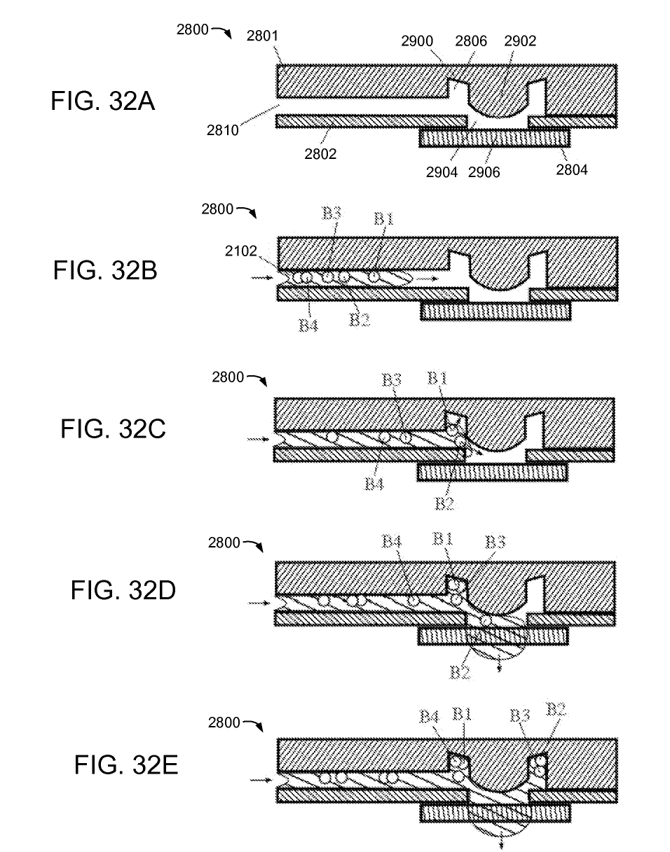

Example operation of system 2800 is described below with respect to FIG. 31A through 31C and FIGS. 32A through 32E.

FIG. 31 depicts movement of fluid that may be a fluid sample, regent fluid or a combination thereof and may contain gaseous bubbles. The active area of the membrane 2804 may contain markers, 3100, that may bind to substances, 3104, in the liquid, 2102, shown in FIG. 31B. The fluid with these substances flow through the membrane and some of them may be captured by the markers. If a gas bubble, 3105, shown in FIG. 31C stays in contact with the membrane, it prevents access of these substances to the markers. Fluid will still flow through the membrane by going around the gas bubble, but the active region may not have full contact.

FIGS. 32A through 32E illustrate example operation of membrane bubble trap system 2800.

In FIGS. 32A through 32C, an active region of membrane 2804, where fluid is to pass through, is positioned over orifice 2904.

Bubble trap system 2800 may be oriented such that upper portion 2801 is opposite to a gravitational pull or centrifugal force, and is substantially level, relative to FIGS. 32A through 32D, such that bubbles travel to bubble termination pathway 2806 by buoyancy forces, and fluid flows downwardly through membrane 2804, such as by gravitational force, centrifugal force, and/or fluid pressure.

In FIG. 32B, fluid 3100 is enters fluid channel 2810. Fluid 3100 may include a fluid sample, reagent fluid, or combination thereof, and may contain gaseous bubbles. For purposes of the instant explanation, four bubbles are depicted and labeled B1, B2, B3, and B4.

In FIG. 32C, when fluid 3100 reaches bubble termination pathway 2806, bubble B1 travels upwardly into the slanted portion of cavity 2900, and bubble B2 is shown as having been forced into cavity 2906, such as by a force of fluid 3100.

In FIG. 32D, bubble B2 may contact membrane 2804, and lower surface 2902 of core portion 2808 may redirect bubble B2 upwardly into bubble termination pathway 2806, as shown in FIG. 32E.

Also in FIG. 32E, bubble B3 has been pushed, relative to FIG. 32D, around bubble termination pathway 2806 to a position opposite bubbles B1 and B4.

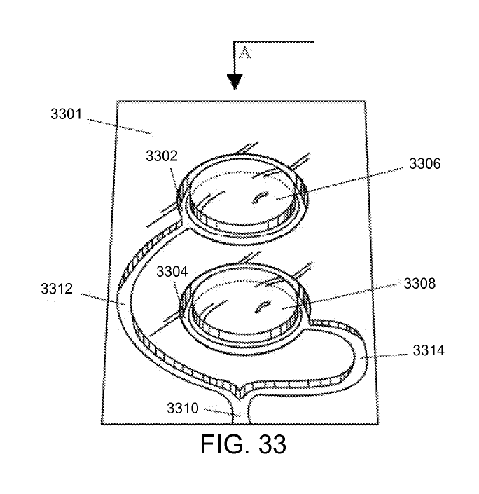

Bubble trap system 2800 may include multiple interconnected membrane active areas, each including a corresponding bubble termination trap. FIG. 33 illustrates an upper portion 3301 including multiple cavities 3302 and 3304 and corresponding curved sections 3306 and 3308.

Upper portion 3301 further includes a fluid channel 3310, including branches 3312 and 3314 to cavities 3302 and 3304.

Branches 3312 and 3314 may have similar fluid resistances and may be of similar length to permit fluid to reach corresponding active areas substantially simultaneously. More than one branch can end at the same bubble termination area.

Bubble trap system 2800 may be implemented within an assay system, such as one or more of assay systems 600, 1500, and 1600. For example, and without limitation, bubble trap system 2800 may be implemented to trap bubbles in an area proximate to a test membrane within assay region 618 in FIG. 6, wherein membrane 2804 of bubble trap system 2800 may be positioned over openings 622, 624, and 626 of assay region 618 in FIG. 6, and upper portion 2801 and lower portion 2802 of bubble trap system 2800, or portions thereof, may be implemented as part of body 602 and/or as part of a cover over assay region 618 of assay system 600. Fluid channel 110 of bubble trap system 100 may correspond to, or extend from fluid passage 730 of assay system 600 in FIG. 7.

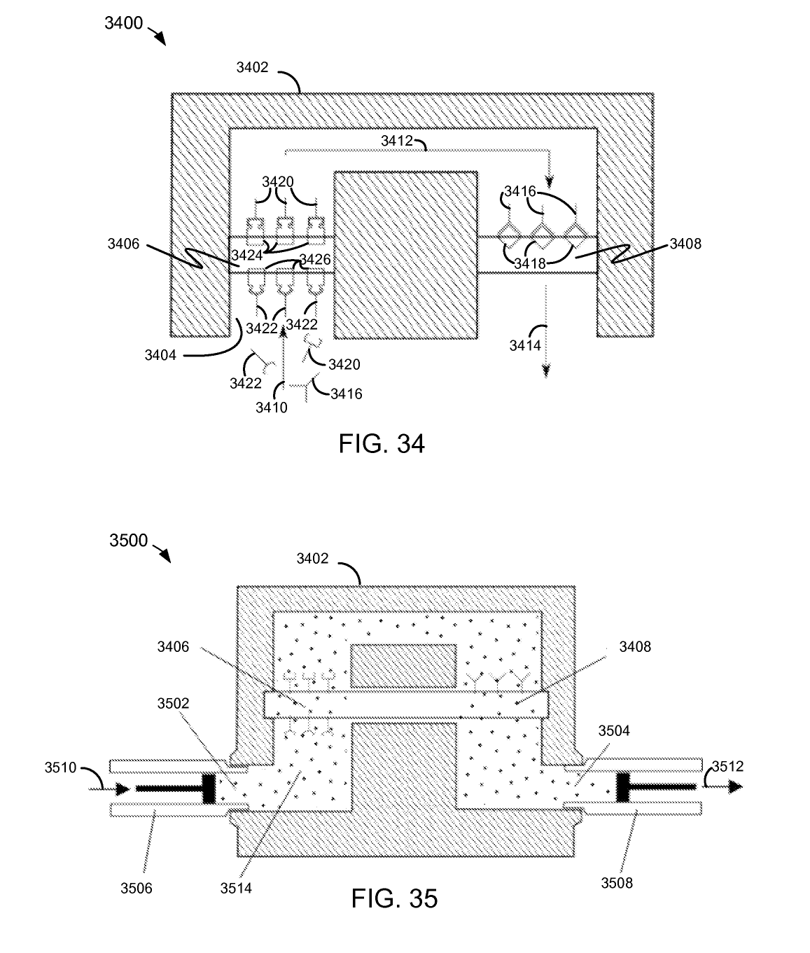

Methods and systems to capture competitive molecules are disclosed below with respect to FIGS. 34-36. The methods and systems may be implemented to capture competitive molecules on a membrane, such as to reduce false positives on a test region of the membrane.

In an embodiment, competitive antibodies, such as antibodies specific to Chlamydia Pneumoniae and Chlamydia Psittaci, may be captured in an assay region to prevent false positives of antibodies specific to Chlamydia Trachomatis.

Antigens specific to both C. Pneumoniae and C. Psittaci may be immobilized in a filter region that a sample passes through before contacting the assay region. This may help to ensure that only antibodies against C. Trachomatis reach the active area on the testing surface.

As used herein, the phrase, “specific to,” may refer to a molecule associated with a condition. For example, and without limitation, the phrase, “an antibody specific to” a condition, such as a disease, may include an antibody generated in response to the condition. The phrase, “an antigen specific to” a condition may include a molecule a molecule that binds relatively strongly to an antibody generated in response to the condition, and may include, for example, part of a cell wall of a pathogen, or a metabolic protein generated and/or excreted by a pathogen.

Because of affinity differences, antibodies against C. Pneumoniae and C. Psittaci may be bound in a filter region up-stream of an assay region, without substantially reducing the C. Trachomatis signal. This may reduce false positives caused by infection with one of these species, which may increase an overall specificity of the test.

Methods and systems to capture competitive molecules disclosed herein may be implemented with respect to self-contained, point-of-care, portable, point-of-care, user-initiated fluidic assay systems.

FIG. 34 is a cross-sectional block-diagram of an competitive molecule capture system 3400 including a structure 3402 having a fluid passage 3404, and one or more porous membranes disposed therein. The porous membranes may include a filter membrane 3406 and a test membrane 3408. Filter membrane 3406 and test membrane 3408 may correspond to portions of a single membrane, or may correspond to separate membranes.

In the example of FIG. 34, fluid flows through fluid passage 3404, filter membrane 220, and test membrane 220 in directions of arrows 3410, 3412, and 3414. The fluid may include a biological sample from a patient.

Structure 3402 may be manufactured of a relatively rigid plastic such as, for example, styrene, polystyrene, nylon, polycarbonate and/or other suitable material.

Filter membrane 3406 and test membrane 3408 may be made of nitrous cellulose or other suitable material that can immobilize targets in a fluid sample that flows through the membrane.

Fluid system 3400 may be implemented to test for presence of a target antibody 3416 within the biological sample. In a test for target antibody 3416, a corresponding antigen 3418 may be immobilized on test membrane 3408, or an active region thereof. The fluid containing the biological sample from the patient is directed through fluid passage 3404 in the direction of arrows 3410, 3412, and 3414. Where the patient sample includes target antibody 3416, target antibody 3416 binds to antigen 3418 at test membrane 3408, in what is referred to herein as a positive test. The binding may be detected and/or rendered observable in accordance with one or more of a variety of techniques.

The patient sample may, however, include one or more other antibodies, illustrated in FIG. 34 as antibodies 3420 and 3422, which may bind relatively weakly to antigen 3418. Such other antibodies are referred to herein as competing antibodies. Competing antibodies, even when only weakly bound to antigen 3418, may result in a false positive or weak false positive.

To reduce and/or prevent false positives from competing antibodies 3420 and 3422, corresponding antigens 3424 and 3426, specific to antibodies 3420 and 3422, respectively, may be immobilized on filter membrane 3406. An antigen is specific to an antibody when the antigen and the antibody bind with one another. Antigens 3424 and 3426 may effectively capture antibodies 3420 and 3422 from the fluid before the fluid reaches test membrane 3408, which may reduce and/or prevent false positives.

System 3400 may be implemented to test for the presence of one or more of a variety of antibodies including, without limitation, an antibody of Chlamydia Trachomatis.

Where system 3400 is implemented to test for an antibody of Chlamydia Trachomatis, target antibody 3416 may correspond to Chlamydia Trachomatis, and antibodies 3420 and 3422 may correspond to Chlamydia Pneumoniae and Chlamydia Psittaci, respectively. Antigen 3418 may be specific to target antibody 3416, and antigens 3424 and 3426 may be specific to antibodies 3420 and 3422, respectively.

FIG. 35 is a cross-sectional side view of another competitive molecule capture system 3500, including features of system 3400. System 3500 includes one or more fluid inlet ports 3502 and fluid outlet ports 3504. System 3500 and may include one or more plungers, illustrated here as an inlet plunger 3506 and an outlet plunger 3508, to move a fluid 3514 in directions of corresponding arrows 3510 and 3512. One or more of plungers 3506 and 3508 may correspond to a plunger as disclosed in one or more examples above. Alternatively, or additionally, one or more of plungers 3506 and 3508 may correspond to syringe.

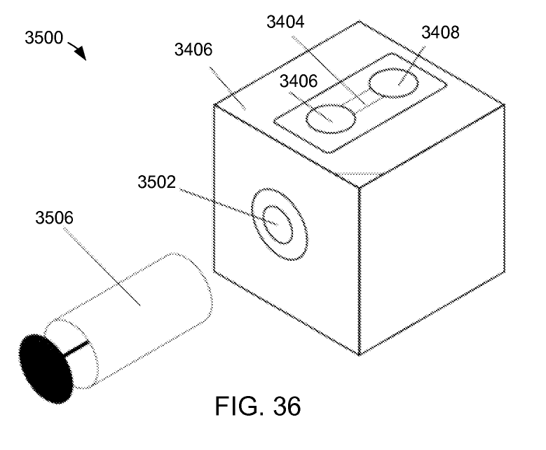

FIG. 36 is a cross-sectional perspective view of system 3500.

Methods and systems to capture competitive antibodies may be implemented to capture one or a plurality of antibodies.

Methods and systems to capture competitive antibodies may be implemented with one or a plurality of target antibodies.

Methods and systems to capture competitive antibodies may be implemented with one or a plurality of inlet and/or outlet fluid passages.

Methods and systems to capture competitive antibodies may be integrated with a system to collect, prepare, and/or assay biological samples, such as one or more methods and systems disclosed herein.

Methods and systems to capture competitive antibodies may be implemented within an assay system, such as one or more of assay systems 600, 1500, and 1600. For example, and without limitation, fluid passage 3404 of system 3400 (FIG. 34) may correspond to a fluid passage between sample region 604 and assay region 618 (FIG. 7), and test membrane 3408 (FIG. 34) may correspond to an assay membrane in assay region 618 (FIG. 7).

Methods and systems disclosed herein, and portions thereof, may be implemented alone and/or in various combinations with one another. For example, and without limitation, one or more of the following, and portions thereof, may be implemented alone, and/or in various combinations and sub-combinations with one another:

methods and systems to implement and operate hand-held portable assays;

methods and systems to activate an assay system;

methods and systems to collect samples;

methods and system to capture antibodies; and

methods and systems to trap or capture gas bubbles.

While various embodiments are disclosed herein, it should be understood that they have been presented by way of example only, and not limitation. It will be apparent to persons skilled in the relevant art that various changes in form and detail may be made therein without departing from the spirit and scope of the methods and systems disclosed herein. Thus, the breadth and scope of the claims should not be limited by any of the example embodiments disclosed herein.

What is claimed is:

1. A system, comprising:
a portable housing having a sample region, the sample region having a fluid inlet and a fluid outlet, the portable housing further having a fluid chamber and a fluid passage from the fluid chamber to the sample region fluid inlet;
a movable cover to enclose the sample region; and
a mechanically actuated fluid controller to force fluid from the fluid chamber, through the fluid passage, the sample region fluid inlet, and the sample region fluid outlet, to move at least a portion of a sample from the sample region through the fluid outlet.

2. The system of claim 1, wherein the movable cover is configured to prevent the mechanically actuated fluid controller from forcing fluid flow when the movable cover is in an open position.

3. The system of claim 1, wherein the sample region is located on an enclosable portion of a surface of the portable housing.

4. The system of claim 1, wherein the moveable cover includes a mechanically slideable cover.

5. The system of claim 4, wherein an outer surface of the portable housing has a substantially cylindrical shape, and wherein the moveable cover includes a slideable sleeve surrounding a portion of the portable housing.

6. The system of claim 1, wherein the mechanically actuated fluid controller includes a mechanical actuator to close the cover and to actuate fluid flow when the movable cover is closed.

7. The system of claim 6, wherein:
the movable cover and the mechanical actuator are releasably coupled to one another; and
the mechanical actuator is configured to close the cover and to thereafter disengage from the cover and actuate fluid flow when the cover is closed.

8. The system of claim 7, further including:
a breakable tab to releasably couple the mechanical actuator to the moveable cover.

9. The system of claim 8, wherein the moveable cover includes a slideable sleeve surrounding a portion of the portable housing.

10. The system of claim 1, wherein the moveable cover includes a hinged cover.

11. The system of claim 1, wherein the moveable cover includes a rotating cover.

12. The system of claim 1, wherein the moveable cover includes a movable window.

13. The system of claim 1, wherein the moveable cover includes a removable window.

14. The system of claim 1, further including:
a sample collection pad disposed within the sample region to receive a biological sample.

15. The system of claim 1, further including:
a sample collection pad disposed within the sample region to receive an environmental sample.

16. The system of claim 1, further including:
a sample collection pad disposed within the sample region to absorb a sample.

17. The system of claim 15, wherein the sample region and the sample collection pad are dimensioned to receive less than approximately 250 micro-liters of the biological sample.

18. The system of claim 1, further including:
a lance device to pierce a membrane to release a biological sample therefrom.

19. The system of claim 1, further including:
a scraping device to scrape a biological sample.

20. The system of claim 1, wherein the mechanically actuated fluid controller is configured to force the fluid from the sample region fluid outlet to a fluid storage chamber.

21. The system of claim 1, wherein the mechanically actuated fluid controller is further configured to seal one or more of the sample region fluid inlet and fluid outlet after the fluid is forced from the sample region fluid outlet to the fluid storage chamber, and to thereafter force another fluid from another fluid chamber to a fluid storage chamber.

22. A sample preparation system comprising:
a sample receiving region that, in a first open position, can receive a sample to be prepared and which, after receiving said sample, in a second closed position, forms a fluid sample chamber;
said fluid sample chamber comprising at least one fluid entry pathway and at least one fluid exit pathway;
a movable plunger to define, in a first position, a fluid reagent chamber containing fluid reagent that can interact with the sample; wherein when said plunger is moved to a second position, forces said fluid reagent from said fluid reagent chamber into said fluid entry pathway, through said fluid sample chamber, out of said fluid exit pathway.

23. The device of claim 22, wherein said fluid exit pathway is configured to connect to an assay system.

24. The device of claim 22, wherein said fluid exit pathway is in fluid communication with a storage chamber to collect said sample and said reagent fluid.

25. The device of claim 24, wherein said storage chamber is physically separate from said sample preparation system.

26. The device of claim 24, wherein said movable plunger is configured to force said fluid reagent from said fluid exit pathway into said storage chamber.

27. The device of claim 22 wherein the storage chamber is in fluid communication with at least one additional fluid chamber in a testing device.

28. The sample preparation system of claim 22 comprising a plurality of fluid sample chamber and fluid reagent chamber pairs operated in parallel by at least one movable plunger

29. The sample preparation system of claim 22 comprising a plurality of fluid reagent chambers, each containing a fluid reagent and all said fluid reagent chambers capable of fluid communication with said fluid sample chamber wherein movement of said movable plunger causes the separate reagent fluids from said plurality of fluid reagent chambers to be all forced through said fluid sample chamber.

30. The device of claim 22 wherein the fluid sample chamber is sealed between two o-rings and a movable hollow cylindrical cover.

31. The device of claim 22 wherein the plunger is a stepped plunger with a smaller diameter section for moving past the sample chamber and forcing reagent fluid into the sample chamber and a larger diameter section for reducing the volume of the fluid sample chamber.

32. A sample preparation system to prepare a sample for testing comprising:
a fluid preparation chamber comprising sample receiving region and a sealing cover having
a first open position wherein said sample may be applied to said sample receiving region, and
a second closed position which prevents further application of sample to said sample receiving region;
wherein said fluid preparation chamber comprises
at least one fluid entry pathway for providing fluid communication between a reagent chamber having a sample preparation reagent and said fluid preparation chamber; and
at least one fluid exit pathway;
a movable plunger which,
when in a first position prevents fluid communication between said fluid preparation chamber and said reagent chamber and through said fluid exit pathway, and
when moved to a second position forces sample preparation reagent from said reagent chamber through said fluid entry pathway into said fluid preparation chamber and into contact with said sample receiving region and out of said fluid preparation chamber through said fluid exit pathway.

33. The sample preparation system of claim 32 wherein said sealing cover is either a sleeve which can slide over said sample receiving region and engage at least one O-ring seal, or a hinged cover which engages a seal surrounding said sample receiving region.

34. The sample preparation system of claim 32 wherein said sealing cover is operably linked to said movable plunger whereby said sealing cover must be in said second closed position before said movable plunger can force said sample preparation reagent from said reagent chamber through said fluid entry pathway into said fluid preparation chamber.

35. The sample preparation system of claim 32 wherein said sample receiving region comprises at least one binding agent for binding at least one unwanted component in said sample and retaining said unwanted component upon movement of said sample and sample preparation reagent out of said fluid preparation chamber.

36. The sample preparation system of claim 32 wherein said at least one binding agent is selected from the group consisting of antibodies for specifically binding a pre-determined unwanted component, specific protein for specifically binding one or more unwanted components and specific nucleic acid fragment for specifically binding one or more unwanted components, and non-specific protein for non-specifically binding one or more unwanted components.

37. The sample preparation system of claim 36 wherein said sample receiving region further comprises finger pricking means for collecting a blood sample on said sample receiving region.

38. The sample preparation system of claim 32 wherein said binding agent is an antibody disclosed herein.

39. The sample preparation system of claim 33 wherein said sample preparation reagent is a reagent disclosed herein.

40. The sample preparation system of claim 32, wherein said sample includes one or more of blood, serum, plasma, semen, urine, feces, saliva, sputum, oral fluid, tissue, mucus, pus, tears, or cerebrospinal fluid.

41. A sample preparation system, comprising:
a sample receiving chamber that has a cover in a first position where sample can be applied;
said cover that can be moved to a second position that prevents further sample from being applied;
at least one fluid entry pathway into said sample chamber;
at least one fluid exit pathway out of said sample chamber;
a fluid communication pathway created between the entry pathway and exit pathway when said cover is moved to said second position;
a movable plunger or element that seals a fluid prep chamber containing fluid that will interact with the sample in a first position;
said plunger or element, when moved, forces said fluid into said entry pathway, through said sample chamber and out of said exit pathway

42. Device of claim 41 wherein the exit pathway can be used to deposit sample and prep solution into another module, such as a test-tube for sample shipment, or device that contains a fluid pathway leading to a test membrane that the sample/prep solution mix can interact with.

43. Device of claim 41 wherein the fluid prep chamber can be subdivided by creating parallel plungers to store separate fluids that are all mixed when forced through the sample chamber.

44. Device of claim 41 wherein the sample application chamber is sealed between two o-rings with a movable hollow cylinder.

45. Device of claim 41 wherein the sample application chamber is sealed

46. Device of claim 41 wherein the plunger is a stepped plunger with the smaller diameter section moving past the sample chamber forcing fluid into the sample chamber and the larger diameter section reducing the volume of the fluid prep chamber.

47. Device of claim 41 where said exit pathway and said entry pathway are the same.

48. A sample preparation system, comprising:
a sample receiving chamber that has a cover in a first position where sample can be applied;
a lancet or other mechanism that punctures a patient's finger when pressed into the sample receiving chamber;
said cover that can be moved to a second position that prevents further sample from being applied;
at least one fluid entry pathway into said sample chamber;
at least one fluid exit pathway out of said sample chamber;
a fluid communication pathway created between the entry pathway and exit pathway when said cover is moved to said second position;
a movable plunger or element that seals a fluid prep chamber containing fluid that will interact with the sample in a first position;
said plunger or element, when moved, forces said fluid into said entry pathway, through said sample chamber, out of said exit pathway.

49. The system of claim 14, wherein the sample includes one or more of blood, serum, plasma, semen, urine, feces, saliva, sputum, oral fluid, tissue, mucus, pus, tears, or cerebrospinal fluid.

50. A method of preparing an assay to test a sample for a primary binding pair molecule, comprising:
identifying a primary binding pair molecule for which to test a sample type;
identifying a corresponding binding pair molecule that binds relatively strongly to the primary binding pair molecule;
identifying a competitive molecule that may exist within the sample type and that binds relatively weakly to the corresponding binding pair molecule;
identifying a capture molecule that binds relatively strongly to the competitive molecule;
immobilizing the corresponding binding pair molecule in an assay region; and
immobilizing the capture molecule in a filter region.

51. The method of claim 50, further including:
receiving a sample of the sample type within a sample region; and
forcing fluid through the sample region, through the filter region, and to the assay region, to move at least a portion of the sample from the sample region, contact and bind the competitive molecule that may exist within the sample with the capture molecule immobilized within the filter region, and contact and bind the primary binding pair molecule of the sample with the corresponding binding pair molecule immobilized within the assay region.

52. The method of claim 51, further including:
identifying a labeled secondary molecule that binds to the primary binding pair molecule; and
immobilizing the labeled secondary molecule in the assay region.

53. The method of claim 50, wherein the sample region and the filter region are located within a sample collection system and the assay region is located within an assay system.

54. The method of claim 53, wherein the sample collection system and the assay system are physically separate from one another.

55. The method of claim 53, wherein the sample collection system and the assay system are implemented within a housing of a portable, point-of-care assay apparatus.

56. The method of claim 50, wherein the capture molecule includes one or more of, an analyte;
an antibody,
an antigen,
an oligonucleotide,
a protein fragment,
a nucleic acid fragment, and
a dissolved gas.

57. The method of claim 50, wherein:
the primary binding pair molecule includes a primary binding pair analyte;
the corresponding binding pair molecule includes a corresponding binding pair capture reagent that binds relatively strongly to the primary binding pair analyte;
the competitive molecule includes a competitive analyte that binds relatively weakly to the corresponding binding pair capture reagent; and
the capture molecule includes a capture reagent that binds relatively strongly to the competitive analyte.

58. The method of claim 57, wherein:
the primary binding pair analyte is specific to a first condition, and
the competitive analyte is specific to a second condition that may co-occur with the first condition.

59. The method of claim 58, wherein:
the primary binding pair analyte includes an antibody specific to the first condition;
the corresponding binding pair capture reagent includes a first antigen that binds relatively strongly with the antibody specific to the first condition;
the competitive analyte includes an antibody specific to the second condition and that binds relatively weakly with the first antigen; and
the capture reagent includes a second antigen that binds relatively strongly with the antibody specific to the second condition.

60. The method of claim 58, wherein:
the primary binding pair analyte includes an antigen specific to the first condition;
the corresponding binding pair capture reagent includes a first antibody that binds relatively strongly with the antigen specific to the first condition;
the competitive analyte includes an antigen specific to the second condition and that binds relatively weakly with the first antibody; and
the capture reagent includes a second antibody that binds relatively strongly with the antigen specific to the second condition.

61. The method of claim 58, wherein the first condition includes Chlamydia trachomatis and the second condition includes one or more of Chlamydia pneumoniae and Chlamydia psittaci.

62. The method of claim 58, wherein the first condition includes one of HSV-1 and HSV-2 (herpes simplex virus type 1 and type 2), and the second condition includes the other of HSV-1 and HSV-2.

63. The method of claim 58, wherein the first condition includes one of HIV-1 and HIV-2, and the second condition includes the other of HIV-1 and HIV-2.

64. The method of claim 58, wherein the first condition includes one of Treponema pallidum and Borrelia burgdorferi, Borrelia afzelii, and Borrelia garinii, and the second condition includes the other of Treponema pallidum and Borrelia burgdorferi, Borrelia afzelii, and Borrelia garinii.

65. The method of claim 58, wherein the first condition includes one of Plasmodium falciparum histidine-rich protein 2 (PfHRP-2) and rheumatoid factor, and the second condition includes the other of Plasmodium falciparum histidine-rich protein 2 (PfHRP-2) and rheumatoid factor.

66. The method of claim 58, wherein the first condition includes one of Trypanosoma cruzi and Trypanosoma rangeli, and the second condition includes the other of Trypanosoma cruzi and Trypanosoma rangeli.

67. The method of claim 58, wherein the first condition includes one of Cardiac troponin I and skeletal troponin I, and the second condition includes the other of Cardiac troponin I and skeletal troponin I.

68. The method of claim 58, wherein the first condition includes one of Luteinizing hormone (LH), follicle-stimulating hormone (FSH), thyroid-stimulating hormone (TSH), and human chorionic gonadotropin (hCG), and the second condition includes one or more other of LH, FSH, TSH, and hCG.

69. A system, comprising:
a sample collection system including a housing having a sample region to receive a sample type, a filter region, a fluid chamber, and a mechanically actuated fluid controller movably disposed within the sample collection housing to force fluid from the fluid chamber, through the sample region, and through the filter region;
an assay system to receive fluid from the filter region, wherein the assay system includes an assay region to test a sample type for a primary binding pair molecule;
a corresponding binding pair molecule that binds relatively strongly to the primary binding pair molecule immobilized within the assay region;
wherein the sample type may include a competitive molecule that binds relatively weakly to the corresponding binding pair molecule; and
a capture system within the filter region to capture the competitive molecule from fluid that passes from the sample region and through the filter region.

70. The apparatus of claim 69, wherein the sample collection system and the assay system are physically separate from one another.

71. The apparatus of claim 69, wherein the sample collection system and the assay system are implemented within a housing of a portable, point-of-care assay apparatus.

72. The apparatus of claim 69, wherein the capture system includes:
a capture molecule that binds relatively strongly to the competitive molecule immobilized within the filter region.

73. The apparatus of claim 72, wherein the capture molecule includes one or more of, an analyte;
an antibody,
an antigen,
an oligonucleotide,
a protein fragment,
a nucleic acid fragment, and
a dissolved gas.

74. The apparatus of claim 69, wherein:
the primary binding pair molecule includes a primary binding pair analyte;
the corresponding binding pair molecule includes a corresponding binding pair capture reagent that binds relatively strongly to the primary binding pair analyte;
the competitive molecule includes a competitive analyte that binds relatively weakly to the corresponding binding pair analyte; and
the capture molecule includes a capture reagent that binds relatively strongly to the competitive analyte.

75. The apparatus of claim 70, wherein:
the primary binding pair analyte is specific to a first condition, and
the competitive analyte is specific to a second condition that may co-occur with the first condition.

76. The apparatus of claim 71, wherein:
the primary binding pair analyte includes an antibody specific to the first condition;
the corresponding binding pair capture reagent includes a first antigen that binds relatively strongly with the antibody specific to the first condition;
the competitive analyte includes an antibody specific to the second condition and that binds relatively weakly with the first antigen; and
the capture reagent includes a second antigen that binds relatively strongly with the antibody specific to the second condition.

77. The apparatus of claim 71, wherein:
the primary binding pair analyte includes an antigen specific to the first condition;
the corresponding binding pair capture reagent includes a first antibody that binds relatively strongly with the antigen specific to the first condition;
the competitive analyte includes an antigen specific to the second condition and that binds relatively weakly with the first antibody; and
the capture reagent includes a second antibody that binds relatively strongly with the antigen specific to the second condition.

78. The apparatus of claim 71, wherein the first condition includes Chlamydia trachomatis and the second condition includes one or more of Chlamydia pneumoniae and Chlamydia psittaci.

79. The apparatus of claim 71, wherein the first condition includes one of HSV-1 and HSV-2 (herpes simplex virus type 1 and type 2), and the second condition includes the other of HSV-1 and HSV-2.

80. The apparatus of claim 71, wherein the first condition includes one of HIV-1 and HIV-2, and the second condition includes the other of HIV-1 and HIV-2.

81. The apparatus of claim 71, wherein the first condition includes one of Treponema pallidum and Borrelia burgdorferi, Borrelia afzelii, and Borrelia garinii, and the second condition includes the other of Treponema pallidum and Borrelia burgdorferi, Borrelia afzelii, and Borrelia garinii.

82. The apparatus of claim 71, wherein the first condition includes one of Plasmodium falciparum histidine-rich protein 2 (PfHRP-2) and rheumatoid factor, and the second condition includes the other of Plasmodium falciparum histidine-rich protein 2 (PfHRP-2) and rheumatoid factor.

83. The apparatus of claim 71, wherein the first condition includes one of Trypanosoma cruzi and Trypanosoma rangeli, and the second condition includes the other of Trypanosoma cruzi and Trypanosoma rangeli.

84. The apparatus of claim 71, wherein the first condition includes one of Cardiac troponin I and skeletal troponin I, and the second condition includes the other of Cardiac troponin I and skeletal troponin I.

85. The apparatus of claim 71, wherein the first condition includes one of Luteinizing hormone (LH), follicle-stimulating hormone (FSH), thyroid-stimulating hormone (TSH), and human chorionic gonadotropin (hCG), and the second condition includes one or more other of LH, FSH, TSH, and hCG.

86. The apparatus of claim 69, further including:
a labeled secondary molecule that binds to the primary binding pair molecule, immobilized within the assay region.

87. A portable, point-of-care, self contained assay system, comprising:
a portable housing having a sample collection region, a sample analyzing region, a plurality of fluid chambers, and a plurality of fluid passages from the fluid chambers to one or more of the sample collection region and the sample analyzing region, wherein fluid outlets of the fluid chambers are movable relative to the fluid passages; and
a mechanically actuated fluid controller to repeatedly reposition the fluid chamber fluid outlets relative to the fluid passages and to sequentially force fluid from each of a plurality of sets of one or more of the fluid chambers, through corresponding fluid chamber outlets, and to one or more of the sample collection region and the sample analyzing region subsequent to at least a subset of the repositionings.

88. The system of claim 87, wherein the mechanically actuated fluid controller is configured to laterally and rotationally reposition the fluid chamber outlets relative to the fluid passages.

89. The system of claim 87, wherein the mechanically actuated fluid controller is configured to laterally and rotationally reposition the fluid chamber outlets relative to the fluid passages in accordance with a sequence of predetermined positions, each including a lateral component and a rotational component.

90. The system of claim 89, wherein each of the predetermined positions includes one of a sequence of rotational positions and an alternating one of a first and a second lateral position.

91. The system of claim 89, wherein the mechanically actuated fluid controller includes:
a control device movable within the portable housing to control movement of the fluid chamber fluid outlets relative to the fluid passages; and
a mechanical actuator;
wherein the control device is configured to sequentially move through the sequence of predetermined positions in response to movement of the mechanical actuator and in accordance with a mechanical pattern imbedded within the system.

92. The system of claim 91, wherein:
the mechanical pattern includes a recessed path within an inner surface of the portable housing;
the control device includes a post extending from the control device to slideably engage the recessed path; and
the recessed path is patterned to guide the control device through the sequence of predetermined positions in response to movement of the mechanical actuator.

93. The system of claim 92, wherein:
the mechanical actuator includes a button extending through a surface of the portable housing; and
the recessed path is patterned to guide the control device through the sequence of predetermined positions in response to lateral movement of the button.

94. The system of claim 93, wherein:
the button is configured to move the control device from a first lateral position to a second lateral position in response to movement of the button from a first position to a second position; and
a compression device to compress in response to movement of the button from the first position to the second position, and to move the control device from the second lateral position to the first lateral position and the button from the second position to the first position when the compression device is compressed;
wherein each of the predetermined positions includes one of a sequence of rotational positions and an alternating one of the first and second lateral positions.

95. The system of claim 94, further including:
a coupling device to restrain the compression device relative to the control device while the button is pressed, and to thereafter release the compression device to move the control device from the second lateral position to the first lateral position and the button from the second position to the first position.

96. The system of claim 92, wherein:
the mechanical actuator includes a mechanically twistable device to rotate the control device relative to the portable housing; and
the recessed path includes a spiral pattern to guide the control device through the sequence of predetermined positions in response to rotational movement of the twistable device, including to rotationally and laterally move the control device relative to the portable housing.

97. The system of claim 87, wherein the mechanically actuated fluid controller includes:
a control device movable within the portable housing to control movement of the fluid chamber fluid outlets relative to the fluid passages; and
a post extending from a the control device to slideably engage a recessed path of an inner surface of the portable housing;
wherein the recessed path is patterned to guide the control device through a sequence of predetermined positions in response to movement of the control device.

98. The system of claim 97, wherein the control device includes a rod extending therefrom to impart the sequence of predetermined positions to the fluid chamber fluid outlets relative to the fluid passages.

99. The system of claim 97, wherein:
the control device is laterally and rotationally movable within the portable housing; and
the recessed path is patterned to guide the control device through the sequence of predetermined positions, each including a lateral component and a rotational component.

100. The system of claim 99, wherein each of the predetermined positions includes one of a sequence of rotational components and an alternating one of a first and a second lateral component.

101. The system of claim 97, wherein the control device includes:
a mechanical button extending through a surface of the portable housing to laterally move the control device.

102. The system of claim 97, wherein the control device includes:
a mechanically twistable device to rotate the control device relative to the portable housing.

103. A method of operating a portable, point-of-care, self contained assay system that includes a portable housing having a sample collection region, a sample analyzing region, a plurality of fluid chambers, and a plurality of fluid passages to one or more of the sample collection region and the reaction region, wherein fluid outlets of the fluid chambers are movable relative to the fluid passages, the method comprising:
repeatedly repositioning the fluid chamber fluid outlets relative to the fluid passages in response to lateral movement of a button extending from the assay system housing; and
sequentially forcing fluid from each of a plurality of sets of one or more of the fluid chambers, through corresponding fluid chamber outlets, and to one or more of the sample collection region and the sample analyzing region subsequent to at least a subset of the repositionings.

104. The method of claim 103, wherein the repositioning includes laterally and rotationally repositioning the fluid chamber outlets relative to the fluid passages.

105. The method of claim 103 wherein the repositioning includes laterally and rotationally repositioning the fluid chamber outlets relative to the fluid passages in accordance with a sequence of predetermined positions, each including a lateral component and a rotational component.

106. The method of claim 105, wherein each of the predetermined positions includes one of a sequence of rotational positions and an alternating one of a first and a second lateral position.

107. The method of claim 105, wherein the repositioning further includes:
sequentially repositioning through the sequence of predetermined positions in accordance with a mechanical pattern imbedded within the assay system.

108. The method of claim 107, wherein the mechanical pattern includes a recessed path within an inner surface of the portable housing and the assay system includes a fluid control device movable within the assay system housing and having a post extending therefrom to slideably engage the recessed path, and wherein the repositioning further includes;
guiding the post through the recessed path to move the control device through the sequence of predetermined positions in response to movement of the fluid control device relative to the assay system housing.

109. The method of claim 108, wherein the repositioning further includes:
repositioning in response to a mechanically operated button coupled to the fluid control device.

110. The method of claim 108, wherein the repositioning further includes:
repositioning in response to a mechanically operated rotational device coupled to the fluid control device.

111. A structure to trap a predetermined amount of moving gas bubbles in a liquid, comprising:
a housing including one or more fluid pathways of predetermined cross-sectional shape and size connecting a source of fluid and an orifice having a predetermined size,
said orifice in fluid communication with a porous membrane,
a bubble collection pathway of predetermined cross-sectional shape and size surrounding said orifice and a central core and in fluid communication with said fluid pathways,
said central core aligned with said orifice and extending below said bubble collection pathway a predetermined distance, but not in contact with said porous membrane,
wherein said orifice and said central core cooperate to direct fluid flow onto said membrane while permitting gas bubbles to flow into and be collected by said bubble collection pathway.

112. The structure of claim 111 wherein said central core comprise optically transparent material selected from the group consisting of nylon, styrene, polystyrene, and polycarbonate.

113. A system, comprising:
a housing including a cavity therein, a first opening from the cavity through a lower surface of the housing, and a second opening from the cavity to a fluid channel to permit a fluid to flow from the fluid channel to the cavity, wherein the fluid channel includes an upwardly directed opening to a bubble pathway to permit bubbles in the fluid to rise into the bubble pathway.

114. The system of claim 113, further including:
a porous material sealing disposed against the lower surface of the housing and over the opening through the lower surface of the housing.

115. The system of claim 113, wherein the housing includes a convex portion disposed over the cavity to cause bubbles in the cavity to rise to the bubble pathway.

116. A method, comprising:
forcing a fluid into a fluid channel of a housing, wherein the housing includes a cavity, a first opening from the cavity through a lower surface of the housing, and a second opening from the cavity to the fluid channel to permit the fluid to flow from the fluid channel to the cavity, and wherein the fluid channel includes an upwardly directed opening to a bubble pathway to permit bubbles in the fluid to rise into the bubble pathway;
trapping gas bubbles from the fluid in the bubble pathway; and
passing the fluid through the opening in the lower surface of the housing.

117. The method of claim 116, wherein a porous material is sealing disposed against the lower surface of the housing and over the opening through the lower surface of the housing, the method further including:
passing the fluid through the porous membrane.

118. A system, comprising:
means for providing a fluid to a cavity of a housing; and
means for trapping gas bubbles from the fluid prior to the bubbles entering the cavity

119. The system of claim 118, further including:
means for directing gas bubbles within the cavity to the means for trapping.

120. The system of claim 118, wherein the housing includes an opening from the cavity through a lower surface of the housing, the system further including:
a porous material sealing disposed against the lower surface of the housing and over the opening through the lower surface of the housing.
